(19) United States
(12) Patent Application Publication
(10) Pub. No.: US 2006/0264463 A1
(43) Pub. Date: Nov. 23, 2006

(54) Chemical compounds

(76) Inventors: Christopher Luckhurst, Leicestershire (GB); Tobias Mochel, Leicestershire (GB); Matthew Perry, Leicestershire (GB); Brian Springthorpe, Leicestershire (GB); Linda Stein, Leicestershire (GB)

Correspondence Address: FISH & RICHARDSON P.C., P.O BOX 1022, MINNEAPOLIS, MN, 55440-1022, US

(21) Appl. No.: 10/556,107

(22) PCT Filed: May 6, 2004
(86) PCT No.: PCT/SE04/00693

§ 371 Date: Nov. 8, 2005

(30) Foreign Application Priority Data
May 9, 2003 (SE) 0301368-7

(51) Int. Cl. C07D 401/14; A61K 31/4545

(52) U.S. Cl. 514/316; 514/318; 546/194; 546/187

(57) ABSTRACT

The present invention provides a compound of a formula (1): wherein the variables are defined herein; to a process for preparing such a compound; and to the use of such a compound in the treatment of a chemokine (such as CCR3) or H1 mediated disease state.

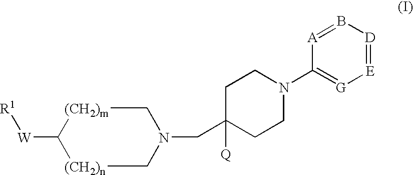

The present invention concerns piperidine derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in WO99/38514, WO99/04794 and WO00/35877.

Histamine is a basic amine, 2-(4imidazolyl)-ethylamine, and is formed from histidine by histidine decarboxylase. It is found in most tissues of the body, but is present in high concentrations in the lung, skin and in the gastrointestinal tract. At the cellular level inflammatory cells such as mast cells and basophils store large amounts of histamine. It is recognised that the degranulation of mast cells and basophils and the subsequent release of histamine is a fundamental mechanism responsible for the clinical manifestation of an allergic process. Histamine produces its actions by an effect on specific histamine G-protein coupled receptors, which are of three main types, H1, H2 and H3. Histamine H1 antagonists comprise the largest class of medications used in the treatment of patients with allergic disorders, especially rhinitis and urticaria. H1 antagonists are useful in controlling the allergic response by for example blocking the action of histamine on post-capillary venule smooth muscle, resulting in decreased vascular permeability, exudation and oedema. The antagonists also produce blockade of the actions of histamine on the H1 receptors on c-type nociceptive nerve fibres, resulting in decreased itching and sneezing.

Chemokines are chemotactic cytokines that are released by a wide variety of cells tot attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or a) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

Viral infections are known to cause lung inflammation. It has been shown experimentally that the common cold increases mucosal output of eotaxin in the airways. Instillation of eotaxin into the nose can mimic some of the signs and symptoms of a common cold. (See, Greiff L et al Allergy (1999) 54(11) 1204-8 [Experimental common cold increase mucosal output of eotaxin in atopic individuals] and Kawaguchi M et al Int. Arch. Allergy Immunol. (2000) 122 S1 44 [Expression of eotaxin by normal airway epithelial cells after virus A infection].)

The present invention provides a compound of formula (I): 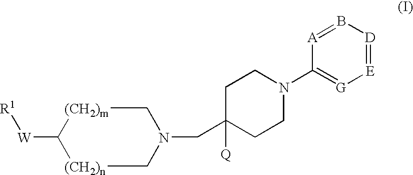
wherein:
n and m are, independently, 0 or 1;
A, B, D, E, G represent one each of CCO₂R⁵, CR², CR³, CR⁴, and CH or N,
Q is hydrogen or hydroxy;
W is CH₂, O, NH or N(C₁₋₄ alkyl);
R₁ is phenyl optionally substituted by halogen, cyano, C(O)NH₂, C₁₋₄ alkyl, C₁₋₄ haloalkyl, C₁₋₄ alkoxy or C₁₋₄ haloalkoxy;
R², R³ and R⁴ are, independently, hydrogen, halogen, cyano, nitro, hydroxy, NR⁶R⁷, C₁₋₆ alkyl (optionally substituted with halogen), C₁₋₆ alkoxy (optionally substituted with halogen), S(O)ₚ(C₁₋₆ alkyl), S(O)_{q}CF₃ or S(O)₂NR⁷R⁸;
R⁵ is hydrogen, C₁₋₆ alkyl or benzyl;
p and q are, independently, 0, 1 or 2;
   R⁶, R⁷, R⁸ and R⁹ are, independently, hydrogen, C₁₋₆ alkyl (optionally substituted by halogen, hydroxy or C₃₋₆ cycloalkyl), CH₂(C₂₋₅ alkenyl), phenyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
alternatively NR⁶R⁷ or NR⁸R⁹ may, independently, form a 4-7 membered heterocyclic ring, azetidine, pyrrolidine, piperidine, azepine, morpholine or piperazine, the latter optionally substituted by C₁₋₄ alkyl on the distal nitrogen;
or an N-oxide thereof; or a pharmaceutically acceptable salt thereof; or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, dihydrochloride, hydrobromide, phosphate, sulfate, acetate, diacetate, fumarate, maleate, tartrate, citrate, oxalate, methanesulfonate or p-toluenesulfonate.

The compounds of the invention may exist as solvates (such as hydrates) and the present invention covers all such solvates.

Halogen includes fluorine, chlorine, bromine and iodine. Halogen is, for example, fluorine or chlorine.

Alkyl groups and moieties are straight or branched chain and comprise, for example, 1 to 6 (such as 1 to 4) carbon atoms. Examples of alkyl groups are methyl, ethyl, n-propyl, iso-propyl or tert-butyl.

Haloalkyl groups and moieties comprise an alkyl part, as defined above, and one or more (for example 1 to 6) of the same or different halogen atoms. Haloalkyl is, for example, CF₃.

Alkenyl groups comprise, for example, 2 to 6 (such as 2 to 4) carbon atoms. Examples of alkenyl groups are vinyl or allyl.

In one embodiment cycloalkyl groups comprise from 3 to 6 carbon atoms and are monocyclic. Cycloalkyl is, for example, cyclopropyl, cyclopentyl or cyclohexyl.

Heterocyclyl is an aromatic or non-aromatic 5 or 6 membered ring, optionally fused to one or more other rings, comprising at least one heteroatom selected from the group comprising nitrogen, oxygen and sulfur; or an N-oxide thereof, or an S-oxide or S-dioxide thereof. Heterocyclyl is, for example, furyl, thienyl (also known as thiophenyl), pyrrolyl, 2,5-dihydropyrrolyl, thiazolyl, pyrazolyl, oxazolyl, isoxazolyl, imidazolyl, piperidinyl, morpholinyl, pyridinyl, dihydropyridinyl (for example in a 6-oxo-1,6-dihydro-pyridinyl moiety), pyrimidinyl, indolyl, 2,3-dihydroindolyl, benzo[b]furyl (also known as benzofuryl), benz[b]thienyl (also known as benzthienyl or benzthiophenyl), 2,3-dihydrobenz[b]thienyl (for example in a 1-dioxo-2,3-dihydrobenz[b]thienyl moiety), indazolyl, benzimidazolyl, benztriazolyl, benzoxazolyl, benzthiazolyl (for example in a 1H-benzthiazol-2-one-yl moiety), 2,3-dihydrobenzthiazolyl (for example in a 2,3-dihydrobenzthiazol-2-one-yl moiety), 1,2,3-benzothiadiazolyl, an imidazopyridinyl (such as imidazo[1,2a]pyridinyl), thieno[3,2-b]pyridin-6yl, 1,2,3-benzoxadiazolyl, benzo[1,2,3]thiadiazolyl, 2,1,3-benzothiadiazolyl, benzofurazan (also known as 2,1,3-benzoxadiazolyl), quinoxalinyl, dihydro-1-benzopyryliumyl (for example in a coumarinyl or a chromonyl moiety), 3,4-dihydro-1H-2,1-benzothiazinyl (for example in a 2-dioxo-3,4-dihydro-1H-2,1-benzothiazinyl moiety), a pyrazolopyridine (for example 1H-pyrazolo[3,4-b]pyridinyl), a purine (for example in a 3,7-dihydro-purin-2,6-dione-8-yl moiety), quinolinyl, isoquinolinyl, dihydroisoquinolinyl (for example in a 2H-isoquinolin-1-one-yl moiety), a naphthyridinyl (for example [1,6]naphthyridinyl or [1,8]naphthyridinyl), a dihydro[1,8]naphthyridinyl (for example in a 1H-[1,8]naphthyridin4-one-yl moiety), a benzothiazinyl, a dihydrobenzothiazinyl (for example in a 4H-benzo[1,4]thiazin-3-one-yl moiety), benzo[d]imidazo[2,1-b]thiazol-2-yl or dibenzothiophenyl (also known as dibenzothienyl); or an N-oxide thereof, or an S-oxide or S-dioxide thereof.

An N-oxide of a compound of formula (I) is, for example, a 1-oxy-[1,4′]bipiperidinyl-1′-yl compound.

In one particular aspect the invention provides a compound of formula (I) 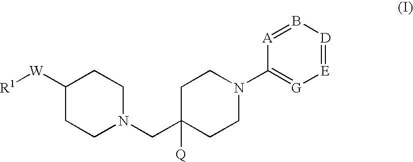
wherein:
n and m are both 1;
A, B, D, E, G represent one each of CCO₂R⁵, CR², CR³, CR⁴, and CH or N;
Q is hydrogen or hydroxy;
W is CH₂, O, NH or N(C₁₋₄ alkyl);
R¹ is phenyl optionally substituted by halogen, cyano, C₁₋₄ alkyl, C₁₋₄ haloalkyl, C₁₋₄ alkoxy or C₁₋₄ haloalkoxy;
R², R³ and R⁴ are, independently, hydrogen, halogen, cyano, nitro, hydroxy, NR⁶R⁷, C₁₋₄ alkyl (optionally substituted with halogen), C₁₋₆ alkoxy (optionally substituted with halogen), S(O)ₚ(C₁₋₆ alkyl), S(O)_{q}CF₃ or S(O)₂NR⁷R⁸;
R⁵ is hydrogen, C₁₋₆ alkyl or benzyl;
p and q are, independently, 0, 1 or 2;
R⁶, R⁷, R⁸ and R⁹ are, independently, hydrogen, C₁₋₆ alkyl (optionally substituted by halogen, hydroxy or C₃₋₆ cycloalkyl), CH₂(C₂₋₅ alkenyl), phenyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
alternatively NR⁶R⁷ or NR⁸R⁹ may, independently, form a 4-7 membered heterocyclic ring, azetidine, pyrrolidine, piperidine, azepine, morpholine or piperazine, the latter optionally substituted by C₁₄ alkyl on the distal nitrogen;
or an N-oxide thereof; or a pharmaceutically acceptable salt thereof; or a solvate thereof In a further aspect the present invention provides a compound wherein W is O.

In a still further aspect the present invention provides a compound of formula (I) where n and m are both 1.

In another aspect R¹ is phenyl optionally substituted (for example independently mono- or di-substituted) with halogen (for example chlorine or fluorine), cyano, C₁₋₄ alkyl (for example methyl) or C₁₋₄ alkoxy (for example methoxy).

In yet another aspect R¹ is phenyl optionally substituted (for example independently mono- or di-substituted) with halogen (for example chlorine or fluorine), C₁₋₄ alkyl (for example methyl) or C₁₋₄ alkoxy (for example methoxy).

In a further aspect R¹ is phenyl optionally substituted (for example with one, two or three of the same or different) with fluorine, chlorine, cyano, C₁₋₄ alkyl (for example methyl) or C₁₋₄ alkoxy (for example methoxy). In a still further aspect R¹ is phenyl substituted by one, two or three (for example two or three) substituents independently selected from: fluorine, chlorine, cyano and methyl. For example R¹ is 3,4-dichlorophenyl, 2,4dichloro-3-methylphenyl, 3,4-dichloro-2-methylphenyl, 2,4dichlorophenyl, 4chloro-2-methylphenyl, 2-chloro-4-fluorophenyl, 4-fluorophenyl or 3-chloro-4-cyanophenyl.

In a still further aspect of the invention Q is hydrogen.

In yet another aspect of the invention R⁵ is hydrogen.

In a further aspect of the invention A is CCO₂R⁵, for example wherein R⁵ is hydrogen or C₁₋₄ alkyl (such as methyl).

In a still further aspect of the invention one of A, B, D, E, G represent one each of CCO₂R⁵, CR², CR³, CR⁴, and CH or N; wherein R², R³ and R⁴, are, independently, hydrogen, halogen, cyano, nitro, C₁₋₄ alkyl (such as methyl or ethyl), C₁₋₄ alkoxy (such as methoxy or ethoxy), CF₃, OCF₃, S(O)₂(C₁₋₄ alkyl) (such as S(O)₂CH₃) or S(O)₂NH₂; and R⁵ is hydrogen or C₁₋₆ alkyl (such as methyl, ethyl or tert-butyl).

In yet another aspect of the invention one of B, D, E and G is CH and the others are CR², CR³ and CR⁴, wherein R², R³ and R⁴, are, independently, hydrogen, halogen, cyano, C₁₋₄ alkyl (such as methyl or ethyl), C₁₋₄ alkoxy (such as methoxy or ethoxy), CF₃, OCF₃, S(O)₂(C₁₋₄ alkyl) (such as S(O)₂CH₃) or S(O)₂NH₂ {for example R², R³ and R⁴, are, independently, hydrogen, halogen, cyano, nitro, C₁₋₄ alkyl (such as methoxy or ethoxy), or C₁₋₄ alkoxy (such as methoxy or ethoxy), CF₃ or OCF₃}. In a further aspect of the invention B, D, E and G are all CH.

In a further aspect of the invention A, B, D, E, G represent one each of CCO₂R⁵, CR², CR³, CR⁴, and CH or N; wherein R², R³ and R⁴ are, independently, hydrogen, halogen (such as fluorine or chlorine), C₁₋₄ alkyl (such as methyl), C₁₋₄ alkoxy (such as methoxy), nitro or CF₃; and R⁵ is hydrogen or C₁₋₆ alkyl (such as methyl, ethyl or tert-butyl).

In another aspect the present invention provides a compound of formula (I) wherein: Q is hydrogen; W is O or CH₂; A is CCO₂R⁵; B, D, E and G are all CH; R¹ is phenyl substituted by halogen (for example by one or two chlorine atoms), cyano or C₁₋₄ alkyl (for example methyl); and R⁵ is hydrogen or C₁₋₄ alkyl (for example methyl).

In a further aspect the present invention provides a compound of formula (I) wherein n and m are, independently, 0 or 1, R¹ is phenyl optionally substituted by halogen (such as fluorine or chlorine), cyano, C₁₋₄ alkyl (such as methyl) or C(O)NH₂; W is O or CH₂; Q is H or OH; A, B, D, E, G represent one each of CCO₂R⁵, CR², CR³, CR⁴, and CH or N; wherein R², R³ and R⁴ are, independently, hydrogen, halogen (such as fluorine or chlorine), C₁₋₄ alkyl (such as methyl), C₁₋₄ alkoxy (such as methoxy), nitro or CF₃; and R⁵ is hydrogen or C₁₋₆ alkyl (such as methyl, ethyl or tert-butyl).

The compounds of the present invention can be prepared as described below.

A compound of formula (I) can be prepared by nucleophilic substitution of fluoride (II): 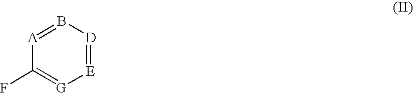
with a compound of formula (III): 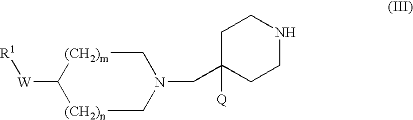
in the presence of a suitable solvent (such as N,N-dimethylformamide), at a suitable temperature (for example in the range 10-100° C., such as 70-90° C.), optionally in the presence of a suitable base (such as potassium carbonate).

For a compound of formula (I):
when R⁵ is hydrogen said compound may be converted to a compound of the invention where R⁵ is not hydrogen by a standard esterification method well known in the art; and,
when R⁵ is not hydrogen said compound may be converted to a compound of the invention where R⁵ is hydrogen by a standard ester hydrolysis method well known in the art.

A compound of formula (III) can be prepared by deprotecting a compound of formula (IV): 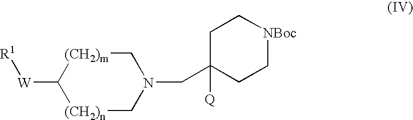
for example using trifluoroacetic acid in a suitable solvent (such as dichloromethane) or using a source of hydrogen chloride in a suitable solvent (such as dioxane).

A compound of formula (IV), wherein Q is hydrogen, can be prepared by reacting a compound of formula (V): 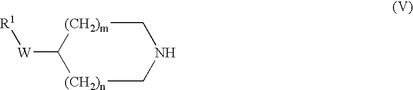
with a compound of formula (VI): 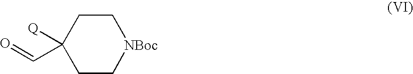
in the presence of NaBH(OAc)₃ and acetic acid, in a suitable solvent (such as tetrahydrofuran or dichloromethane).

A compound of formula (IV), wherein Q is hydroxy, can be prepared by reacting a compound of formula (V) with a compound of formula (VII): 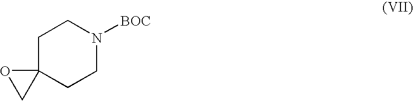
in a suitable solvent (such as a C₁₋₆ aliphatic alcohol, for example ethanol) at room temperature.

Alternatively, a compound of formula (I) can be prepared by a Buchwald-Hartwig amination, where a compound of formula (VIII): 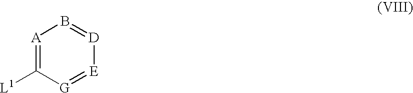
wherein L¹ is bromo or iodo; is reacted with a compound of formula (III) in the presence of a suitable palladium compound (such as tris-(dibenzylidineacetone)dipalladium), a ligand (such as 2-dicyclohexylphosphino-2′-(N,N-dimethylamino)biphenyl), a base (such as caesium carbonate) and a solvent (such as a solvent comprising a ether, for example dioxin) at a suitable temperature (such as in the range 80-100° C.).

The preparation of various intermediates can be found in WO00/66559 and WO01/77101; alternatively they can be prepared by using or adapting literature methods.

Further compounds of formula (I) can be prepared by adaptation of: the routes described above, methods described in the art or the Examples recited below.

Compounds of formula (II) to (VII) can be prepared by using or adapting methods described in the art. The preparation of various phenoxy piperidines is described in WO 01/77101.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of chemokine receptor (especially CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these conditions are:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata, corneal ulcer or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), peridontal disease, Sezary syndrome, idiopathic thrombocytopenia pupura or disorders of the menstrual cycle.

The compounds of formula (I) or a pharmaceutically acceptable salt thereof or a solvate thereof, are also H1 antagonists (and can, therefore, be used in the treatment of allergic disorders); and may also be used to control a sign and/or symptom of what is commonly referred to as a cold (for example a sign and/or symptom of a common cold or influenza or other associated respiratory virus infection).

According to a further feature of the present invention there is provided a method for treating a chemokine mediated disease state (especially a CCR3 mediated disease state) in a mammal, such as man, suffering from, or at risk of, said disease state, which comprises administering to a mammal in need of such treatment a therapeutically effective amount of a compound of the formula (I) or a pharmaceutically acceptable salt thereof or a solvate thereof.

According to another feature of the present invention there is provided a method for antagonising H1 in a mammal, such as man, suffering from, or at risk of, an H1 mediated disease state, which comprises administering to a mammal in need of such treatment a therapeutically effective amount of a compound of the formula (I) or a pharmaceutically acceptable salt thereof or a solvate thereof.

According to yet another feature of the present invention there is provided a method for treating a sign and/or symptom of what is commonly referred to as a cold in a mammal, such as man, suffering from, or at risk of, said disease state, which comprises administering to a mammal in need of such treatment a therapeutically effective amount of a compound of the formula (I) or a pharmaceutically acceptable salt thereof or a solvate thereof.

The invention also provides a compound of the formula (I), or a pharmaceutically acceptable salt thereof or a solvate thereof, for use in therapy.

In another aspect the invention provides the use of a compound of formula (I), or a pharmaceutically acceptable salt thereof or a solvate thereof, in the manufacture of a medicament for use in therapy (for example modulating chemokine receptor activity (especially CCR3 receptor activity), antagonising H1 or treating a sign and/or symptom of what is commonly referred to as a cold).

The invention further provides the use of a compound of formula (I), or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systemic sclerosis;
(3) (skin and eyes) psoriasis, atopic dermatitis, contact dermatitis or other eczmatous dermitides, seborrhoetic dermatitis, lichen planus, phemphigus, bullous phemphigus, epidermolysis bullosa, urticaria, angiodermas, vasculitides erythemas, cutaneous eosinophilias, uveitis, alopecia areata, corneal ulcer or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;
   in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of formula (I), or a pharmaceutically acceptable salt thereof, is useful in the treatment of asthma.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In order to use a compound of the invention, or a pharmaceutically acceptable salt thereof or solvate thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof or a solvate thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (per cent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.01 mgkg⁻¹ to 100 mgkg⁻¹, such as in the range of 0.1 mgkg⁻¹ to 20 mgkg⁻¹, of the active ingredient administered, for example, 1 to 4 times per day.

The invention further relates to combination therapies wherein a compound of formula (1) or a pharmaceutically acceptable salt, solvate or in vivo hydrolysable ester thereof, or a pharmaceutical composition or formulation comprising a compound of formula (1) is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents such as: Non-steroidal anti-inflammatory agents (hereinafter NSAIDs) including non-selective cyclo-oxygenase (COX)-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate, leflunomide; hydroxychloroquine, d-penicillamine, auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

The present invention still further relates to the combination of a compound of the invention together with modulators of chemokine receptor function such as antagonists of CCR1, CCR2, CCR2A, CCR2B, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention still further relates to the combination of a compound of the invention together with an inhibitor of matrix metalloproteases ( s), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-1) and MMP-9 and MMP-12, including agents such as doxycycline.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; N-(5-substituted)-thiophene-2-alkylsulfonamides; 2,6-di-tert-butylphenolhydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a phosphodiesterase (PDE) inhibitor such as the methylxanthines including theophylline and aminophylline; and selective PDE isoenzyme inhibitors including PDE4 inhibitors and inhibitors of the isoform PDE4D, and inhibitors of PDE5.

The present invention still further relates to the combination of a compound of the invention together with histamine type 1 receptor antagonists such as cetirizine, loratadine, desloratadine, fexofenadine, acrivastine, terfenadine, astemizole, azelastine, levocabastine, chlorpheniramine, promethazine, cyclizine, and mizolastine applied orally, topically or parenterally.

The present invention still further relates to the combination of a compound of the invention together with a proton pump inhibitor (such as omeprazole) or gastroprotective histamine type 2 receptor antagonist.

The present invention still further relates to the combination of a compound of the invention with antagonists of the histamine type 4 receptor.

The present invention still further relates to the combination of a compound of the invention together with an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride, and ethylnorepinephrine hydrochloride.

The present invention still further relates to the combination of a compound of the invention together with anticholinergic agents including muscarinic receptor (M1, M2, and M3) antagonists such as atropine, hyoscine, glycopyrrrolate, ipratropium bromide, tiotropium bromide, oxitropium bromide, pirenzepine, and telenzepine.

The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol, including chiral enantiomers thereof.

The present invention still further relates to the combination of a compound of the invention together with a chromone, including sodium cromoglycate and nedocromil sodium.

The present invention still further relates to the combination of a compound of the invention together with a glucocorticoid, such as flunisolide, triamcinolone acetonide, beclomethasone dipropionate, budesonide, fluticasone propionate, ciclesonide, and mometasone furoate.

The present invention still further relates to the combination of a compound of the invention together with an agent that modulate nuclear hormone receptors such as PPARs.

The present invention still further relates to the combination of a compound of the invention together with an immunoglobulin (Ig) or Ig preparation or an antagonist or antibody modulating Ig function such as anti-IgE (e.g. omalizumab).

The present invention still further relates to the combination of a compound of the invention together with other systemic or topically-applied anti-inflammatory agents including thalidomide and derivatives, retinoids, dithranol, and calcipotriol.

The present invention still further relates to the combination of a compound of the invention together with combinations of aminosalicylates and sulfapyridine such as sulfasalazine, mesalazine, balsalazide, and olsalazine; and immunomodulatory agents such as the thiopurines, and corticosteroids such as budesonide.

The present invention still further relates to the combination of a compound of the invention together with an antibacterial agent including penicillin derivatives, tetracyclines, macrolides, beta-lactams, fluoroquinolones, metronidazole, and inhaled aminoglycosides; and antiviral agents including acyclovir, famciclovir, valaciclovir, ganciclovir, cidofovir; amantadine, rimantadine; ribavirin; zanamavir and oseltamavir; protease inhibitors such as indinavir, nelfinavir, ritonavir, and saquinavir; nucleoside reverse transcriptase inhibitors such as didanosine, lamivudine, stavudine, zalcitabine, zidovudine; non-nucleoside reverse transcriptase inhibitors such as nevirapine, efavirenz.

The present invention still further relates to the combination of a compound of the invention together with cardiovascular agents such as calcium channel blockers, beta-adrenoceptor blockers, angiotensin-converting enzyme (ACE) inhibitors, angiotensin-2 receptor antagonists; lipid lowering agents such as statins, and fibrates; modulators of blood cell morphology such as pentoxyfylline; thrombolytics, and anticoagulants including platelet aggregation inhibitors.

The present invention still further relates to the combination of a compound of the invention together with CNS agents such as antidepressants (such as sertraline), anti-Parkinsonian drugs (such as deprenyl, L-dopa, ropinirole, pramipexole, MAOB inhibitors such as selegine and rasagiline, comP inhibitors such as tasmar, A-2 inhibitors, dopamine reuptake inhibitors, NMDA antagonists, nicotine agonists, dopamine agonists and inhibitors of neuronal nitric oxide synthase), and anti-Alzheimer's drugs such as donepezil, rivastigmine, tacrine, COX-2 inhibitors, propentofylline or metrifonate.

The present invention still further relates to the combination of a compound of the invention together with agents for the treatment of acute and chronic pain, including centrally and peripherally-acting analgesics such as opioid analogues and derivatives, carbamazepine, phenytoin, sodium valproate, amitryptiline and other antidepressant agents, paracetamol, and non-steroidal anti-inflammatory agents.

The present invention still further relates to the combination of a compound of the invention together with parenterally or topically-applied (including inhaled) local anaesthetic agents such as lignocaine and analogues.

The compounds of the present invention may also be used in combination with anti-osteoporosis agents including hormonal agents such as raloxifene, and biphosphonates such as alendronate.

The present invention still further relates to the combination of a compound of the invention together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) Kinase inhibitors including but not limited to inhibitors of tyrosine kinases (such as Btk, Itk, Jak3 MAP examples of inhibitors might include Gefitinib, Imatinib mesylate), Serine/threonine kinases (including but not limited to inhibitors of MAP kinases such as p38, JNK, protein kinases A, B and C and IKK), and kinases involved in cell cycle regulation (such as but not limted to the cylin dependent kinases); (viii) glucose-6 phosphate dehydrogenase inhibitors; (ix) kinin-B₁- and B₂-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) tachykinin NK₁ and NK₃ receptor antagonists such as the group consisting of NKP-608C; SB-233412 (talnetant); and D-4418; (xx) elastase inhibitors such as the group consisting of UT-77 and ZD-0892; (xxi) TNF-alpha converting enzyme inhibitors (TACE); (xxii) induced nitric oxide synthase (iNOS) inhibitors or (xxiii) chemoattractant receptor-homologous molecule expressed on TH2 cells, (such as CRTH2 antagonists) (xxiv) inhibitors of P38 (xxv) agents modulating the function of Toll-like receptors (TLR) and (xxvi) agents modulating the activity of purinergic receptors such as P2X7; (xxvii) inhibitors of transcription factors activation such as NFkB, API, and STATS.

The compounds of the invention can also be used in combination with existing therapeutic agents for the treatment of cancer. Suitable agents to be used in combination include:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab and the anti-erbb1 antibody cetuxinab [C225]) , farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab, compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:
(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for n/z are given, generally only ions which indicate the parent mass are reported, and unless otherwise stated the mass ion quoted is the positive mass ion—(M+H)⁺;
(iii) the title and sub-title compounds of the examples and methods were named using the name program from Advanced Chemistry Development Inc version 6.0;
(iv) unless stated otherwise, reverse phase HPLC was conducted using a Symmetry™, NovaPak™ or Xerra™ reverse phase silica column; and

(v) the following abbreviations are used:

| | | | |
|---|---|---|---|
| RPHPLC | Reverse phase HPLC | DMSO | Dimethylsulfoxide |
| HPLC | high pressure liquid | aq | Aqueous |
| | chromatography | | |
| TFA | Trifluoroacetic acid | RT | room temperature |
| TMEDA | Tetramethylethylenediamine | THF | Tetrahydrofuran |

### Intermediate 1

This illustrates the preparation of 4-(3,4-dichlorophenoxy)-1-(piperidin4-ylmethyl)piperidine

a) tert-butyl 4-{[4-(3,4-dichlorophenoxy)piperidin-1-yl]methyl}piperidine-1-carboxylate

4-(3,4-Dichlorophenoxy)piperidine (1.27 g) was dissolved in tetrahydrofuran (20 mL); acetic acid (0.5 mL) and tert-butyl 4-formylpiperidine-1-carboxylate (1.43 g) were added to the solution. The reaction mixture was stirred at room temperature for 30 min then sodium triacetoxyborohydride (1.53 g) was added and the mixture was stirred at room temperature overnight. The reaction mixture was poured into 2M sodium hydroxide solution (50 mL) and product was extracted with diethyl ether. The combined ether extracts were washed with brine, dried, filtered and evaporated. Crude material was purified by flash chromatography, (eluting with 979:20:1 dichloromethane:methanol:aqueous ammonia) to give the sub-title compound (2.15 g).

MS 443/445 [M+H)⁺ (ES+)

¹H NMR δ (CDCl₃) 1.06 (2H, ddd), 1.45 (9H, s), 1.61-1.82 (5H, m), 1.92-1.98 (2H, m), 2.16-2.27 (4H, m), 2.65-2.73 (4H, m), 4.08 (2H, d), 4.25 (1H, dq), 6.75 (1H, dd), 6.99 (1H, d), 7.30 (1H, d)

b) 4-(3,4-Dichlorophenoxy)-1-(piperidin4-ylmethyl)-piperidine

tert-butyl 4-{[4-(3,4-dichlorophenoxy)piperidin-1-yl]methyl}piperidine-1-carboxylate (1.0 g) was added to a mixture of 20% TFA in dichloromethane (20 mL) and the mixture was stirred at room temperature for I h. Solvent was removed by evaporation and 2M sodium hydroxide solution (25 mL) was added to the residue. The product was extracted with ethyl acetate and the organic phase was washed with brine, dried, filtered and evaporated to give the title compound (0.5 g).

MS 343/345 [M+H]⁺ (ES+)

¹H NMR δ (CDCl₃) 1.10 (2H, qd), 1.60 (1H, qquintet), 1.73-1.83 (4H, m), 1.90-2.01 (2H, m), 2.16-2.26 (4H, m), 2.55-2.70 (4H, m), 3.09 (2H, d), 4.24 (1H, dquintet), 6.75 (1H, dd), 6.99 (1H, d), 7.27 (1H, d)

The following Intermediates were prepared analogously from the appropriate aryloxy piperidine or arylmethylpiperidine:

| Intermediate | Name (M+H) | ¹H NMR δ (CDCl₃) |
|---|---|---|
| 2 | 4-(2,4-Dichloro-3- | 1.13-1.27(2H, m), 1.57-1.70(1H, |
| | methylphenoxy)-1-(piperidin- | m), 1.76-2.00(2H, m), |
| | 4-ylmethyl)-piperidine | 2.16-2.32(4H, m), 2.46(3H, s), 2.60-2.99(8H, |
| | (357/359) | m), 3.16(2H, d), 4.31(1H, quintet), |
| | | 6.75(1H, d), 7.18(1H, d) |
| 3 | 4-(4-Chloro-2- | 1.08-1.21(2H, m), 1.56-1.68(1H, |
| | methylphenoxy)-1-(piperidin- | m), 1.73-1.86(4H, m), |
| | 4-ylmethyl)-piperidine | 1.90-1.99(2H, m), 2.16-2.31(7H, m), |
| | (322/324) | 2.57-2.69(4H, m), 3.12(2H, d), |
| | | 4.23-4.31(1H, m), 6.74(1H, d), 7.06(1H, dd), |
| | | 7.11(1H, d) |
| 4 | 4-(3,4-Dichloro-2- | (CD₃OD) 1.10-1.22(2H, m), |
| | methylphenoxy)-1-(piperidin- | 1.66-1.85(5H, m), 1.94-2.04(2H, m), |
| | 4-ylmethyl)-piperidine | 2.22(2H, d), 2.31(3H, s), 2.32-2.41(2H, |
| | (357/359) | m), 2.59-2.72(4H, m), 3.08(2H, d), |
| | | 4.38-4.46(1H, m), 6.91(1H, d), |
| | | 7.27(1H, d) |
| 5 | 2-Chloro-4-[[1-(piperidin-4- | (CD₃OD) 1.21-1.32(2H, m), |
| | ylmethyl)-4-piperidinyl]oxy]- | 1.74-1.90(5H, m), 1.99-2.10(2H, m), |
| | benzonitrile (333/335) | 2.26(2H, d), 2.31-2.40(2H, m), |
| | | 2.67-2.79(4H, m), 3.11-3.21(2H, m), |
| | | 4.52-4.62(1H, m), 7.05(1H, dd), 7.21(1H, |
| | | d), 7.70(1H, d) |
| 6 | 4-[(4-Fluorophenyl)methyl]-1- | (CD₃OD+DMSO) 1.19-1.32(4H, m), |
| | (piperidin-4-ylmethyl)- | 1.46-1.54(1H, m), 1.55-1.62(2H, |
| | piperidine ((291) | m), 1.77-1.84(1H, m), |
| | Prepared from 4- | 1.85-1.93(4H, m), 2.17(2H, d), 2.51(2H, d), |
| | fluorobenzylpiperidine | 2.80-2.89(4H, m), 3.23-3.26(2H, m), |
| | (Bioorganic & medicinal | 7.01(2H, t), 7.16(2H, dd) |
| | chemistry letters, 2000, 10, | |
| | 1377) | |
| 7 | 4-(4-Chloro-3- | 1.07-1.21(2H, m), 1.63-1.80(5H, |
| | methylphenoxy)-1-(piperidin- | m), 1.94-2.01(2H, m), 2.21(2H, d), |
| | 4-ylmethyl)piperidine | 2.25-2.34(5H, m), 2.58(2H, td), |
| | (322/324) | 2.66-2.75(2H, m), 2.99-3.06(2H, m), |
| | | 4.30-4.38(1H, m), 6.73(1H, dd), |
| | | 6.84(1H, d), 7.19(1H, d) |
| 8 | 4-(3,4-Dichlorobenzyl)-1- | 7.33(1H, d), 7.23(1H, s), 6.96(1H, d), |
| | (piperidin-4- | 3.06(2H, d), 2.83(2H, d), 2.58(2H, t), |
| | ylmethyl)piperidine (341/343) | 2.47(2H, d), 2.11(2H, d), |
| | | 1.84-1.68(4H, m), 1.64-1.52(3H, m), |
| | | 1.51-1.41(1H, m), 1.31-1.21(2H, m), |
| | | 1.12-1.02(2H, m) |
| 9 | 2-Chloro-3-methyl-4-{[1- | 1.07-1.23(2H, m), 1.64-1.91(5H, |
| | (piperidin-4- | m), 2.01-2.11(2H, m), 2.25(2H, d), |
| | ylmethyl)piperidin-4- | 2.34(3H, s), 2.37-2.47(2H, m), |
| | yl]oxy}benzonitrile | 2.57-2.75(4H, m), 3.02-3.11(2H, m), |
| | (347/349) | 4.58-4.66(1H, m), 7.09(1H, d), 7.62(1H, |
| | | d) |
| 10 | 4-(4-Chlorophenoxy)-1- | |
| | (piperidin-4- | |
| | ylmethyl)piperidine | |
| | (309/311; retention time 1.66, | |
| | standard) | |
| 11 | 3-Chloro-4-[[1-(piperidin-4- | 1.21-1.32(2H, m), 1.74-1.90(5H, |
| | ylmethyl)-4-piperidinyl]oxy]- | m), 1.99-2.10(2H, m), 2.26(2H, d), |
| | benzonitrile (333/335) | 2.31-2.40(2H, m), 2.67-2.79(4H, |
| | | m), 3.11-3.21(2H, m), |
| | | 4.52-4.62(1H, m), 7.05(1H, dd), 7.21(1H, d), |
| | | 7.70(1H, d) |

### Intermediate 12

This illustrates the preparation of 2-chloro-4-(piperidin-4-yloxy)benzonitrile

4-Piperidinol (6.5 g) was added to a solution of potassium tert-butoxide (7.2 g) in N-methyl-2-pyrrolidinone (25 mL) and stirred at room temperature for 30 min. The reaction mixture was cooled to 0° C. and 2-chloro-4-fluoro-benzonitrile (10 g) was added portionwise. The reaction mixture was allowed to reach room temperature and was stirred overnight. LC/MS showed a major peak with the required product mass. The reaction was quenched by addition of ammonium chloride solution and the mixture was partitioned partitioned between ethyl acetate and water. The ethyl acetate was washed with water then brine, dried (MgSO₄), filtered and concentrated in vacuo. Crude material was purified by flash chromatography, eluting with 2-5% methanol, 0.1% triethylamine in dichloromethane to give the title compound (6.6 g).

MS (APCI+ve) 236/238

¹H NMR δ (CD₃OD) 2.00-2.29 (4H, m), 3.22-3.47 (4H, m), 4.85-4.92 (1H, m), 7.14 (1H, dd), 7.33 (1H, d), 7.76 (1H, d).

The following Intermediate was prepared analogously from 2-chlorofluoro-3-methylbenzonitrile

| Intermediate | Name (M+H) | ¹H NMR δ (CD₃OD) |
|---|---|---|
| 13 | 2-chloro-3-methyl-4- | 1.58-1.68(2H, m), 1.89-1.98(2H, |
| | (piperidin-4- | m), 2.22(3H, s), 2.63-2.72(2H, m), |
| | yloxy)benzonitrile | 2.94-3.01(2H, m), 4.53-4.62(1H, |
| | (Retention time | m), 6.99(1H, d), 7.49(1H, d), |
| | 1.62, standard; | |
| | 251/253) | |

### Intermediate 14

This illustrates the preparation of 2-chloro-4-fluoro-3-methylbenzonitrile

2-Chloro-4-fluorobenzonitrile (1 g) and TMEDA (1.13 mL) in THF (10 mL) were cooled to −78° C., under nitrogen. Sec-butyllithium (1.3M in cyclohexane, 8.54 mL) was added over 20 min, keeping the temperature below −70° C. The mixture was then stirred at −78° C. for 2.5 h. Methyl iodode (0.5 mL) was added and the mixture allowed to warm to 15° C., over 35 min. The reaction was quenched with a saturated aqueous solution of ammonium chloride and the product was extracted with ethyl acetate. The ethyl acetate was washed with brine, dried (MgSO4), filtered and concentrated under reduced pressure to give crude product (1.28 g).

Retention time 2.12 min (standard)

### EXAMPLE 1

This Example illustrates the preparation of methyl 2-[4-[[4-(3,4-dichlorophenoxy)-1-piperidinyl]methyl]-1-piperidinyl)-benzoate

4-(3,4-Dichlorophenoxy)-1-(piperidin-4-ylmethyl)-piperidine (0.2 g) and methyl 2-fluorobenzoate (0.08 g) were dissolved in DMF (1 mL) and to the solution was added potassium carbonate (0.1 g). The reaction mixture was heated at 85° C. overnight then poured into water and extracted with ethyl acetate. The ethyl acetate was washed with brine, dried (MgSO₄), filtered and concentrated in vacuo to give a crude yellow oil. This was purified by RPHPLC to give the title compound (0.15 g).

¹H NMR δ (CD₃OD) 1.36-1.51 (2H, m), 1.66-1.89 (5H, m), 1.99-2.09 (2H, m), 2.33-2.46 (4H, m), 2.70-2.84 (4H, m), 3.26-3.30 (2H, m), 3.83 (3H, s), 4.38-4.48 (1H, m), 6.92 (1H, dd), 7.00 (1H, t), 7.11-7.15 (2H, m), 7.38-7.47 (2H, m), 7.64 (1H, dd)

MS (APCI+ve) 476/478

Examples 2-21 in Table 1 were prepared analogously to Example 1 using the appropriate arylfluoride and amine substrates following the general method of Example 1.

### EXAMPLE 22

Methyl 2-(4-{(4-(3,4-dichlorophenoxy)piperidin-1-yl]methyl}piperidin-1-yl)nicotinate

Methyl 2-chloronicotinate (202 mg), 4-(3,4-Dichlorophenoxy)-1-(4-piperidinylmethyl)-piperidine (230 mg) and MP carbonate (Argonaut Technologies Inc) (412 mg) were combined in dimethylacetamide (4 ml) and heated to 120° C. for 5 minutes in a microwave oven (CEM discover). The mixture was filtered and added to water and ethyl acetate. The phases were separated, and the aqueous layer was extracted twice with ethyl acetate. The organic phases were washed with brine, water and brine again, they were then dried, filtered and evaporated. The residue was chromatographed eluting with dichloromethane:methanol (49:1) to give the title compound (125 mg).

MS (ES+ve) 478/480 (M+H)⁺; retention time 2.79 (standard).

### EXAMPLE 23

Ethyl 3-(4-{[4-(3,4-dichlorophenoxy)piperidin-1-yl]methyl}piperidin-1-yl)benzoate

A mixture of 4-(3,4-dichlorophenoxy)-1-(4-piperidinylmethyl)-piperidine (350 mg), ethyl 3-iodobenzoate (161 mg), tris-(dibenzylidineacetone)dipalladium (4.2 mg), caesium carbonate (378 mg), 2-dicyclohexylphosphino-2′-(N,N-dimethylamino)biphenyl (7 mg) in 2-methylpropan-2-ol (7 ml) and dioxan (7 ml) was heated to 95° C. for 1 h. Additional ethyl 3-iodobenzoate (138 mg) was added and heating was continued for 3 h. The mixture was allowed to cool and was then poured into water; the resultant mixture was extracted thrice with ethyl acetate, the organic extracts were combined, washed with brine, dried, filtered and evaporated. Chromatography of the residue (eluent 1:1 isohexane:ethyl acetate) gave the title compound (336 mg).

MS (ES+ve) 491/493 (M+H)⁺

Examples 24-32 in Table 1 were prepared analogously to Example 23 using the appropriate aryl bromide or iodide and amine substrates.

### EXAMPLE 33

This Example illustrates the preparation of 2-[4-[[4-(3,4-dichlorophenoxy)-1-piperidinyl]methyl]-1-piperidinyl]-benzoic acid

Methyl 2-[4-[[4-(3,4-dichlorophenoxy)-1-piperidinyl]methyl]-1-piperidinyl]-benzoate (0.11 g) was dissolved in THF (1 mL) and treated with potassium trimethylsilanolate (36 mg). The reaction mixture was stirred at room temperature overnight then diluted with methanol and acidified to pH 5 by addition of a few drops of acetic acid. The crude solution was purified by RPHPLC to give the title compound (0.080 g).

¹H NMR δ (CD₃OD) 1.40-1.56 (2H, m), 1.64-1.76 (2H, m), 1.87-1.99 (3H, m), 2.06 (2H, d), 2.28-2.38 (4H, m), 2.67-2.76 (2H, m), 3.25-3.32 (4H, m), 4.28-4.38 (1H, m), 6.80 (1H, dd), 7.01 (1H, d), 7.29 (1H, d), 7.38-7.44 (1H, m), 7.54-7.64 (2H, m), 8.08-8.10 (1H, m)

MS (APCI+ve) 460/462

The Examples in Table 2 were prepared analogously to Example 33 by hydrolysis of the corresponding ester using a standard method. In some cases the esters were not isolated and are not included in the table above; such esters were prepared by analogous methods to those in the table above. In the case of Examples 38 and 94 the nitrile in the starting ester also hydrolysed under the reaction conditions.

TABLE 1

| Example | | retention | HPLC | | |
|---|---|---|---|---|---|
| No. | Name | time | system ester | M+H | ¹H NMR |
| 2 | Methyl 2-(4-{[4-(3-chloro-4- | | | 466/468 | (CD₃OD) 1.35-1.50(2H, m), 1.53-1.74(2H, m), |
| | cyanophenoxy)piperidin-1- | | | | 1.76-1.90(4H, m), 2.00-2.12(1H, m), |
| | yl]methyl}piperidin-1- | | | | 2.26-2.43(2H, m), 2.69-2.80(4H, m), 3.25-3.30(4H, |
| | yl)benzoate | | | | m), 3.85(3H, s), 4.53-4.62(1H, m), 6.99(1H, t), |
| | | | | | 7.06(1H, d), 7.13(1H, d), 7.21(1H, d), 7.43(1H, |
| | | | | | t), 7.64(1H, d), 7.70(1H, d) |
| 3 | Methyl 2-(4-{[4-(4- | | | 425/427 | |
| | fluorobenzyl)piperidin-1- | | | | |
| | yl]methyl}piperidin-1- | | | | |
| | yl)benzoate | | | | |
| 4 | Methyl 4-chloro-2-(4-{[4-(3- | | | 502/504 | δ_{(CD3OD)} 1.33-1.51(3H, m), 1.52-1.89(6H, m), |
| | chloro-4- | | | +ve | 2.00-2.11(2H, m), 2.29-2.43(3H, m), |
| | cyanophenoxy)piperidin-1- | | | | 2.69-2.81(4H, m), 3.48(1H, d), 3.89(3H, s), |
| | yl]methyl}piperidin-1- | | | | 4.51-4.63(1H, m), 6.97(1H, dd), 7.01-7.09(2H, m), |
| | yl)benzoate | | | | 7.19-7.22(1H, m), 7.68(2H, dd) |
| 5 | Methyl 2-[4-({4-[4-cyano-2- | | | 468/470 | δ_{(CD3OD)} 1.35-1.50(2H, m), 1.63-1.79(1H, m), |
| | chlorophenoxy]piperidin-1- | | | +ve | 1.82-1.96(5H, m), 2.01-2.13(3H, m), 2.34(2H, |
| | yl}methyl)piperidin-1- | | | | d), 2.41-2.51(2H, m), 2.70-2.81(4H, m), |
| | yl]benzoate | | | | 3.86(3H, s), 4.65-4.74(1H, m), 6.96-7.04(1H, m), |
| | | | | | 7.13(1H, d), 7.28(1H, dd), 7.39-7.48(1H, m), |
| | | | | | 7.62-7.68(2H, m), 7.81(1H, t) |
| 6 | Ethyl 4-(4-{[4-(3,4- | | | 491/493 | |
| | dichlorophenoxy)piperidin-1- | | | | |
| | yl]methyl}piperidin-1- | | | | |
| | yl)benzoate | | | | |
| 7 | Methyl 4-chloro-2-(4-{[4-(2- | | | 500/502 | δ_{(CD3OD)} 1.34-1.45(2H, m), 1.64-1.76(1H, m), |
| | chloro-4- | | | −ve | 1.81-1.91(4H, m), 2.00-2.09(2H, m), 2.30(2H, |
| | cyanophenoxy)piperidin-1- | | | | d), 2.39-2.47(2H, m), 2.68-2.78(4H, m), |
| | yl]methyl}piperidin-1- | | | | 3.25-3.28(2H, m), 3.87(3H, s), 4.64-4.71(1H, m), |
| | yl)benzoate | | | | 6.94(1H, dd), 7.05(1H, d), 7.26(1H, d), |
| | | | | | 7.61-7.66(2H, m), 7.77(1H, d) |
| 8 | Methyl 3-chloro-4-(4-{[4-(3- | 3.02 | standard | 502/504 | |
| | chloro-4- | | | | |
| | cyanophenoxy)piperidin-1- | | | | |
| | yl]methyl}piperidin-1- | | | | |
| | yl)benzoate | | | | |
| 9 | Methyl 2-chloro-4-(4-{[4-(3- | 3.02 | standard | 502/504 | |
| | chloro-4- | | | | |
| | cyanophenoxy)piperidin-1- | | | | |
| | yl]methyl}piperidin-1- | | | | |
| | yl)benzoate | | | | |
| 10 | Methyl 2-(4-{[4-(3,4- | 3.16 | standard | 491/493 | |
| | dichlorophenoxy)piperidin-1- | | | | |
| | yl]methyl}piperidin-1-yl)-4- | | | | |
| | methyl benzoate | | | | |
| 11 | 2-(4-{[4-(4- | 2.64 | standard | | |
| | chlorophenoxy)piperidin-1- | | | | |
| | yl]methyl}piperidin-1- | | | | |
| | yl)benzoate | | | | |
| 12 | Methyl 3-chloro-2-(4-{[4-(3- | 2.9 | standard | 457/459 | |
| | chloro-4- | | | | |
| | cyanophenoxy)piperidin-1- | | | | |
| | yl]methyl}piperidin-1- | | | | |
| | yl)benzoate | | | | |
| 13 | Methyl 2-(4-{[4-(3,4- | 3.18 | standard | 493/495 | |
| | dichlorophenoxy)piperidin-1- | | | | |
| | yl]methyl}-4- | | | | |
| | hydroxypiperidin-1- | | | | |
| | yl)benzoate | | | | |
| 14 | Methyl 2-(4-{[4-(4- | 2.33 | standard | 439/441 | |
| | fluorobenzyl)piperidin-1- | | | | |
| | yl]methyl}piperidin-1-yl)-5- | | | | |
| | methylbenzoate | | | | |
| 15 | Methyl 2-(4-{[3-(3,4- | | | 437/439 | δ_{(CD3OD)} 1.29-1.57(3H, m), 1.79(2H, d), |
| | dichlorophenoxy)azetidin-1- | | | −ve | 2.55(2H, d), 2.70(2H, t), 3.17-3.28(4H, m), |
| | yl]methyl}piperidin-1-yl)-5- | | | | 3.83-3.91(5H, m), 4.81-4.85(1H, m), 6.81(1H, dd), |
| | fluorobenzoate | | | | 7.02(1H, d), 7.15-7.21(2H, m), 7.35-7.40(1H, |
| | | | | | m), 7.43(1H, d) |
| 16 | Methyl 2-(4-{[3-(3,4- | | | 419/421 | δ_{(CD3OD)} 1.35-1.57(3H, m), 1.75-1.84(2H, m), |
| | dichlorophenoxy)azetidin-1- | | | −ve | 2.55(2H, d), 2.73(2H, t), 3.21-3.28(4H, m), |
| | yl]methyl}piperidin-1- | | | | 3.83-3.90(5H, m), 4.80-4.86(1H, m), 6.81(1H, dd), |
| | yl)benzoate | | | | 6.96-7.03(2H, m), 7.12(1H, dd), 7.40-7.47(2H, |
| | | | | | m), 7.64(1H, dd) |
| 17 | Methyl 2-(4-{[(3S)-3-(4- | | | 427/429 | δ_{(CD3OD)} 1.35-1.51(2H, m), 1.57-1.71(1H, m), |
| | chlorophenoxy)pyrrolidin-1- | | | −ve | 1.81-2.00(3H, m), 2.28-2.62(4H, m), |
| | yl]methyl}piperidin-1- | | | | 2.69-2.97(5H, m), 3.25-3.30(2H, m), 3.89(3H, s), |
| | yl)benzoate | | | | 4.86-4.92(1H, m), 6.88(2H, dd), 6.99(1H, t), |
| | | | | | 7.12(1H, dd), 7.26(2H, dd), 7.43(1H, t), |
| | | | | | 7.64(1H, d) |
| 18 | Methyl 2-(4-{[(3S)-3-(4- | | | 445/447 | δ_{(CD3OD)} 1.34-1.49(2H, m), 1.56-1.70(1H, m), |
| | chlorophenoxy)pyrrolidin-1- | | | −ve | 1.81-2.00(3H, m), 2.28-2.62(4H, m), |
| | yl]methyl}piperidin-1-yl)-5- | | | | 2.67-2.96(5H, m), 3.18-3.27(2H, m), 3.90(3H, s), |
| | fluorobenzoate | | | | 4.86-4.92(1H, m), 6.88(2H, dd), 7.16-7.21(2H, |
| | | | | | m), 7.26(2H, dd), 7.35-7.39(1H, m) |
| 19 | Methyl 2-(4-{[3-(3-chloro-4- | | | 438/440 | δ_{(CD3OD)} 1.30-1.59(3H, m), 1.80(2H, d), |
| | cyanophenoxy)azetidin-1- | | | −ve | 2.54(2H, d), 2.73(2H, t), 3.24-3.32(3H, m), |
| | yl]methyl}piperidin-1- | | | | 3.83-3.90(5H, m), 4.92-5.01(2H, m), 6.94-7.03(2H, |
| | yl)benzoate | | | | m), 7.10-7.14(2H, m), 7.43(1H, dd), 7.64(1H, |
| | | | | | dd), 7.74(1H, d) |
| 20 | Methyl 2-(4-{[4-(3-chloro-4- | 2.29 min | standard | 480/482 | |
| | cyano-2- | | gradient | −ve | |
| | methylphenoxy)piperidin-1- | | | | |
| | yl]methyl}piperidin-1- | | | | |
| | yl)benzoate | | | | |
| 21 | Methyl 2-(4-{[3-(3-chloro-4- | 2.64 min | standard | 456/458 | |
| | cyanophenoxy)azetidin-1- | | gradient | −ve | |
| | yl]methyl}piperidin-1-yl)-5- | | | | |
| | fluorobenzoate | | | | |
| 24 | Ethyl 3-(4-{[4-(3,4-dichloro- | | | 505/509 | |
| | 2-methylphenoxy)piperidin-1- | | | | |
| | yl]methyl}piperidin-1- | | | | |
| | yl)benzoate | | | | |
| 25 | 1,1Dimethylethyl 3-(4-{[4- | 3.22 | fast | 533/535 | |
| | (3,4- | | | | |
| | dichlorophenoxy)piperidin-1- | | | | |
| | yl]methyl}piperidin-1-yl)-4- | | | | |
| | methylbenzoate | | | | |
| 26 | 1,1Dimethylethyl 4-chloro-3- | 3.35 | fast | 555/557 | |
| | (4-{[4-(3,4- | | | | |
| | dichlorophenoxy)piperidin-1- | | | | |
| | yl]methyl}piperidin-1- | | | | |
| | yl)benzoate | | | | |
| 27 | 1,1Dimethylethyl 3-(4-{[4- | 2.63 | fast | 549/551 | |
| | (3,4- | | | | |
| | dichlorophenoxy)piperidin-1- | | | | |
| | yl]methyl}piperidin-1-yl)-4- | | | | |
| | methoxybenzoate | | | | |
| 28 | 1,1Dimethylethyl 3-(4-{[4- | | | 505/507 | |
| | (2,4-dichloro-3- | | | | |
| | methylphenoxy)piperidin-1- | | | | |
| | yl]methyl}piperidin-1- | | | | |
| | yl)benzoate | | | | |
| 29 | 1,1Dimethylethyl 2-chloro-5- | 2.91 | fast | 553/555/557 | |
| | (4-{[4-(3,4- | | | | |
| | dichlorophenoxy)piperidin-1- | | | | |
| | yl]methyl}piperidin-1- | | | | |
| | yl)benzoate | | | | |
| 30 | 1,1Dimethylethyl 6-(4-{[4- | 2.43 | fast | 520/522 | |
| | (3,4- | | | | |
| | dichlorophenoxy)piperidin-1- | | | | |
| | yl]methyl}piperidin-1- | | | | |
| | yl)pyridine-2-carboxylate | | | | |
| 31 | 1,1Dimethylethyl 5-(4-{[4- | 1.90 | fast | 534/536 | |
| | (3,4- | | | | |
| | dichlorophenoxy)piperidin-1- | | | | |
| | yl]methyl}piperidin-1-yl)-2- | | | | |
| | methylnicotinate | | | | |
| 32 | Ethyl 3-(4-{[4-(3,4- | 1.43 | fast | 489/491 | |
| | dichlorobenzyl)piperidin-1- | | | | |
| | yl]methyl}piperidin-1- | | | | |
| | yl)benzoate | | | | |

TABLE 2

| | | | MS | | | |
|---|---|---|---|---|---|---|
| | | Hydrolysis | (M+H)⁺ ES | retention | HPLC | |
| Example | Name | Method | +ve | time | system | ¹H NMR δ_{(CD3OD+Idrop NaOD)} |
| 34 | 2-(4-{[4-(3-chloro-4- | A | 452/454 | | | 1.52-1.68(2H, m), 1.78-1.91(2H, m), |
| | cyanophenoxy)piperidin-1- | | −ve | | | 1.98-2.22(5H, m), 2.43(2H, d), |
| | yl]methyl}piperidin-1- | | | | | 2.45-2.52(2H, m), 2.78-2.88(2H, m), |
| | yl)benzoic acid | | | | | 3.36-3.43(4H, m), 4.55-4.65(1H, m), 7.06(1H, dd), |
| | | | | | | 7.22(1H, d), 7.54(1H, dd), 7.67-7.76(3H, |
| | | | | | | m), 8.19-8.22(1H, m) |
| 35 | 2-(4-{[4-(4- | A | 411/413 | | | 1.27-1.45(3H, m), 1.50-1.75(5H, m), |
| | fluorobenzyl)piperidin-1- | | | | | 1.97-2.13(3H, m), 2.26(2H, s), |
| | yl]methyl}piperidin-1- | | | | | 2.51-2.59(4H, m), 3.10-3.17(2H, m), obscured: |
| | yl)benzoic acid | | | | | 3.32-3.39(3H, m), 6.96-7.03(2H, m), |
| | | | | | | 7.14-7.20(2H, m), 7.43-7.49(1H, m), 7.64(2H, |
| | | | | | | d), 8.12(1H, d) |
| 36 | 4-chloro-2-(4-{[4-(3-chloro- | A | 488/490 | | | 1.49-1.69(2H, m), 1.86-2.24(9H, m), |
| | 4-cyanophenoxy)piperidin- | | | | | 2.57-2.78(4H, m), 2.92-3.07(2H, m), |
| | 1-yl]methyl}piperidin-1- | | | | | 3.15-3.30(2H, m), 4.64-4.76(1H, m), |
| | yl)benzoic acid | | | | | 7.05-7.15(1H, m), 7.23-7.31(1H, m), |
| | | | | | | 7.40-7.49(1H, m), 7.67-7.79(2H, m), |
| | | | | | | 8.01-8.10(1H, m) |
| 37 | 2-[4-({4-[4- | A | 472/474 | | | 1.39-1.51(2H, m), 1.58-1.70(1H, m), |
| | (aminocarbonyl)-2- | | | | | 1.76-1.91(4H, m), 1.99-2.08(2H, m), |
| | chlorophenoxy]piperidin-1- | | | | | 2.29(2H, d), 2.37-2.45(2H, m), |
| | yl}methyl)piperidin-1- | | | | | 2.64-2.77(4H, m), 3.46-3.51(2H, m), |
| | yl]benzoic acid | | | | | 4.58-4.66(1H, m), 6.85(1H, td), 6.95(1H, d), |
| | | | | | | 7.13-7.19(2H, m), 7.32(1H, dd), 7.80(1H, dd), |
| | | | | | | 7.94(1H, d) |
| 38 | 4-(4-{[4-(3,4- | A | 463/ | | | 1.28-1.42(2H, m), 1.73-1.86(3H, m), |
| | dichlorophenoxy)piperidin- | | 465(M+H) | | | 1.91(2H, d), 2.00-2.10(2H, m), 2.30(2H, |
| | 1-yl]methyl}piperidin-1- | | | | | d), 2.38(2H, t), 2.72-2.84(4H, m), |
| | yl)benzoic acid | | | | | 3.83(2H, d), 4.44(1H, dd), 6.94(3H, dd), |
| | | | | | | 7.11(1H, d), 7.42(1H, d), 7.88(2H, d) |
| 39 | 4-chloro-2-(4-{[4-(2-chloro- | A | 486/488 | | | 1.48-1.64(2H, m), 1.91-2.20(7H, m), |
| | 4-cyanophenoxy)piperidin- | | −ve | | | 2.55(2H, d), 2.64-2.75(2H, m), |
| | 1-yl]methyl}piperidin-1- | | | | | 2.87-2.98(2H, m), 3.15-3.27(2H, m), |
| | yl)benzoic acid | | | | | 3.35-3.41(2H, m), 4.72-4.82(1H, m), 7.30(1H, d), |
| | | | | | | 7.42(1H, d), 7.65-7.70(2H, m), 7.82(1H, |
| | | | | | | dd), 8.02(1H, d) |
| 40 | 3-chloro-4-(4-{[4-(3-chloro- | B | 488/490 | 1.28 | Standard | 1.36-1.50(2H, m), 1.63-1.91(5H, m), |
| | 4-cyanophenoxy)piperidin- | | | | | 1.98-2.10(2H, m), 2.27-2.42(4H, m), |
| | 1-yl]methyl}piperidin-1- | | | | | 2.62-2.81(4H, m), 3.36-3.46(2H, m), |
| | yl)benzoic acid | | | | | 4.51-4.60(1H, m), 7.04(1H, dd), 7.07(1H, |
| | | | | | | d), 7.19(1H, d), 7.69(1H, d), 7.80(1H, dd), |
| | | | | | | 7.92(1H, d) |
| 41 | 2-chloro-4-(4-{[4-(3-chloro- | B | | 1.17 | Standard | 1.15-1.27(2H, m), 1.58-1.82(5H, m), |
| | 4-cyanophenoxy)piperidin- | | | | | 1.90-1.99(2H, m), 2.17(2H, d), |
| | 1-yl]methyl}piperidin-1- | | | | | 2.22-2.31(2H, m), 2.58-2.69(4H, m), |
| | yl)benzoic acid | | | | | 3.59-3.67(2H, m), 4.42-4.50(1H, m), 6.74(1H, dd), |
| | | | | | | 6.78(1H, d), 6.94(1H, dd), 7.09(1H, d), |
| | | | | | | 7.34(1H, d), 7.59(1H, d) |
| 42 | 2-(4-{[4-(3,4- | B | 477/479 | 2.07 | Standard | 1.28-1.41(2H, m), 1.46-1.59(1H, m), |
| | dichlorophenoxy)piperidin- | | | | | 1.62-1.73(4H, m), 1.86-1.96(2H, m), |
| | 1-yl]methyl}piperidin-1-yl)- | | | | | 2.12-2.28(7H, m), 2.52-2.60(2H, m), |
| | 4-methylbenzoic acid | | | | | 2.60-2.69(2H, m), 3.33-3.40(2H, m), |
| | | | | | | 4.25-4.33(1H, m), 6.57(1H, d), 6.65(1H, |
| | | | | | | s), 6.79(1H, dd), 7.00(1H, d), 7.14(1H, d), |
| | | | | | | 7.28(1H, d) |
| 43 | 2-(4-{[4-(4- | B | 443/445 | 1.84 | Standard | 1.37-1.55(2H, m), 1.58-1.70(1H, m), |
| | chlorophenoxy)piperidin-1- | | | | | 1.74-1.86(4H, m), 1.97-2.09(2H, m), |
| | yl]methyl}piperidin-1-yl)-4- | | | | | 2.25-2.39(7H, m), 2.62-2.84(4H, m), |
| | methylbenzoic acid | | | | | 3.43-3.54(2H, m), 4.32-4.43(1H, m), |
| | | | | | | 6.66-6.80(2H, m), 6.89-6.98(2H, m), |
| | | | | | | 7.21-7.31(3H, m) |
| 44 | 2-(4-{[4-(4- | B | | 2.64 | Standard | 1.39-1.56(2H, m), 1.58-1.72(1H, m), |
| | chlorophenoxy)piperidin-1- | | | | | 1.75-1.87(4H, m), 1.96-2.09(2H, m), |
| | yl]methyl}piperidin-1- | | | | | 2.26-2.41(4H, m), 2.64-2.83(4H, m), |
| | yl)benzoic acid | | | | | 3.45-3.57(2H, m), 4.33-4.43(1H, m), |
| | | | | | | 6.82-6.99(4H, m), 7.13-7.37(4H, m) |
| 45 | 2-(4-{[4-(3-chloro-4- | A | 523/525 | 1.52 | Standard | 1.23-1.37(2H, m), 1.71-1.86(4H, m), |
| | cyanophenoxy)piperidin-1- | | | | | 1.98-2.09(2H, m), 2.24(2H, d), |
| | yl]methyl}piperidin-1-yl)-6- | | | | | 2.30-2.40(2H, m), 2.70(2H, s), 2.92(2H, t), 3.30(1H, |
| | (trifluoromethyl)nicotinic | | | | | s), 4.14(2H, d), 4.51-4.58(1H, m), |
| | acid | | | | | 6.96(1H, d), 7.04(1H, dd), 7.14(1H, d), |
| | | | | | | 7.67-7.75(2H, m) |
| 46 | 2-(4-{[4-(3-chloro-4- | A | 499/501 | | | 1.33-1.52(2H, m), 1.76-1.89(5H, m), |
| | cyanophenoxy)piperidin-1- | | | | | 2.00-2.11(2H, m), 2.28-2.44(4H, m), |
| | yl]methyl}piperidin-1-yl)-5- | | | | | 2.71-2.81(2H, m), 2.93-3.05(2H, m), |
| | nitrobenzoic acid | | | | | 3.75-3.85(2H, m), 4.52-4.64(1H, m), |
| | | | | | | 6.98(1H, d), 7.06(1H, dd), 7.21(1H, d), |
| | | | | | | 7.71(1H, d), 8.05(1H, dd), 8.24(1H, d) |
| 47 | 2-(4-{[4-(3,4- | A | 478/476 | 1.75 | standard | 1.35(2H, q), 1.69-1.82(5H, m), |
| | dichlorophenoxy)piperidin- | | | | | 1.96-2.04(2H, m), 2.25(2H, d), 2.29-2.37(2H, m), |
| | 1-yl]methyl}piperidin-1-yl)- | | | | | 2.34(3H, s), 2.68-2.77(2H, m), 2.87(2H, |
| | 6-methylnicotinic acid | | | | | t), 3.98(2H, d), 4.33-4.43(1H, m), |
| | | | | | | 6.54(1H, d), 6.86-6.91(1H, m), 7.07(1H, d), |
| | | | | | | 7.37(1H, d), 7.56(1H, d) |
| 48 | 3-chloro-2-(4-{[4-(3-chloro- | A | 488/490 | | | 1.21-1.36(2H, m), 1.55-1.77(5H, m), |
| | 4-cyanophenoxy)piperidin- | | | | | 1.89-2.00(2H, m), 2.16-2.31(4H, m), |
| | 1-yl]methyl}piperidin-1- | | | | | 2.59-2.69(2H, m), 3.06-3.13(4H, m), |
| | yl)benzoic acid | | | | | 4.41-4.50(1H, m), 6.83(1H, t), 6.94(1H, |
| | | | | | | dd), 7.07-7.10(2H, m), 7.15(1H, dd), |
| | | | | | | 7.59(1H, d) |
| 49 | 2-(4-{[4-(4-chloro-2- | B | 442/444 | 1.99 | Standard | 1.34-1.51(2H, m), 1.57-1.69(1H, m), |
| | methylphenoxy)piperidin-1- | | | | | 1.74-1.86(4H, m), 1.94-2.06(2H, m), |
| | yl]methyl}piperidin-1- | | | | | 2.18(3H, s), 2.25-2.41(4H, m), |
| | yl)benzoic acid | | | | | 2.62-2.77(4H, m), 3.44-3.52(2H, m), |
| | | | | | | 4.34-4.42(1H, m), 6.83-6.89(2H, m), |
| | | | | | | 6.93-6.96(1H, m), 7.06-7.11(2H, m), |
| | | | | | | 7.13-7.18(1H, m), 7.32(1H, dd) |
| 50 | 2-(4-{[4-(3-chloro-4- | A | 522/524 | | | 1.39-1.54(2H, m), 1.63-1.75(1H, m), |
| | cyanophenoxy)piperidin-1- | | | | | 1.79-1.89(4H, m), 2.00-2.12(2H, m), |
| | yl]methyl}piperidin-1-yl)-4- | | | | | 2.31(2H, d), 2.34-2.43(2H, m), |
| | (trifluoromethyl)benzoic | | | | | 2.71-2.81(4H, m), 3.52-3.60(2H, m), |
| | acid | | | | | 4.53-4.62(1H, m), 7.06(1H, dd), 7.14(2H, d), |
| | | | | | | 7.21(1H, d), 7.46(1H, d), 7.71(1H, d) |
| 51 | 5-chloro-2-(4-{[4-(3-chloro- | A | 488/490 | | | 1.37-1.52(2H, m), 1.57-1.72(1H, m), |
| | 4-cyanophenoxy)piperidin- | | | | | 1.75-1.88(4H, m), 2.00-2.10(2H, m), |
| | 1-yl]methyl}piperidin-1- | | | | | 2.30(2H, d), 2.37(2H, t), 2.63-2.80(4H, |
| | yl)benzoic acid | | | | | m), 3.43-3.51(2H, m), 4.53-4.62(1H, m), |
| | | | | | | 6.93(1H, d), 7.06(1H, dd), 7.16(1H, dd), |
| | | | | | | 7.21(1H, d), 7.30(1H, d), 7.71(1H, d) |
| 52 | 2-(4-{[4-(3-chloro-4- | A | 522/524 | | | 1.38-1.53(2H, m), 1.64-1.89(5H, m), |
| | cyanophenoxy)piperidin-1- | | | | | 2.01-2.12(2H, m), 2.31(2H, d), 2.38(2H, |
| | yl]methyl}piperidin-1-yl)-5- | | | | | t), 2.71-2.86(4H, m), 3.59-3.67(2H, m), |
| | (trifluoromethyl)benzoic | | | | | 4.54-4.63(1H, m), 7.01-7.08(2H, m), |
| | acid | | | | | 7.21(1H, d), 7.44(1H, dd), 7.62(1H, d), |
| | | | | | | 7.71(1H, d) |
| 53 | 2-(4-{[4-(3-chloro-4- | A | 472/474 | | | 1.29-1.47(2H, m), 1.59-1.88(5H, m), |
| | cyanophenoxy)piperidin-1- | | | | | 2.00-2.11(2H, m), 2.29(2H, d), 2.37(2H, |
| | yl]methyl}piperidin-1-yl)-3- | | | | | t), 2.71-2.80(2H, m), 3.14(2H, t), |
| | fluorobenzoic acid | | | | | 3.23-3.32(2H, m), 4.52-4.62(1H, m), |
| | | | | | | 6.87-7.09(4H, m), 7.21(1H, d), 7.71(1H, d) |
| 54 | 2-(4-{[4-(3-chloro-4- | A | 472/474 | | | 1.37-1.52(2H, m), 1.57-1.72(1H, m), |
| | cyanophenoxy)piperidin-1- | | | | | 1.76-1.88(4H, m), 2.00-2.11(2H, m), |
| | yl]methyl}piperidin-1-yl)-5- | | | | | 2.30(2H, d), 2.32-2.43(2H, m), |
| | fluorobenzoic acid | | | | | 2.61-2.81(4H, m), 3.37-3.45(2H, m), |
| | | | | | | 4.52-4.62(1H, m), 6.88-7.09(4H, m), 7.21(1H, d), |
| | | | | | | 7.71(1H, d) |
| 55 | 2-(4-{[4-(3-chloro-4- | A | 484/486 | | | 1.31-1.42(2H, m), 1.56-1.72(3H, m), |
| | cyanophenoxy)piperidin-1- | | | | | 1.75-1.85(2H, m), 1.99-2.07(2H, m), |
| | yl]methyl}piperidin-1-yl)-3- | | | | | 2.26(2H, d), 2.31-2.39(2H, m), |
| | methoxybenzoic acid | | | | | 2.68-2.77(2H, m), 3.11-3.21(4H, m), 3.80(3H, s), |
| | | | | | | 4.52-4.59(1H, m), 6.81(2H, d), 6.97(1H, |
| | | | | | | t), 7.04(1H, dd), 7.18(1H, d), 7.69(1H, d) |
| 56 | 2-(4-{[4-(3-chloro-4- | A | 468/470 | | | 1.37-1.48(2H, m), 1.56-1.65(1H, m), |
| | cyanophenoxy)piperidin-1- | | | | | 1.73-1.84(4H, m), 1.99-2.08(2H, m), |
| | yl]methyl}piperidin-1-yl)-5- | | | | | 2.23(3H, s), 2.27(2H, d), 2.31-2.39(2H, |
| | methylbenzoic acid | | | | | m), 2.63(2H, dd), 2.69-2.77(2H, m), |
| | | | | | | 3.39-3.45(2H, m), 4.52-4.59(1H, m), 6.85(1H, |
| | | | | | | d), 6.98(1H, dd), 7.01-7.05(1H, m), |
| | | | | | | 7.14(1H, d), 7.18(1H, d), 7.69(1H, d) |
| 57 | 2-(4-{[4-(4-chloro-2- | A | 461/463 | | | 1.32-1.48(2H, m), 1.61-1.89(5H, m), |
| | methylphenoxy)piperidin-1- | | | | | 1.97-2.08(2H, m), 2.21(3H, s), 2.29(2H, |
| | yl]methyl}piperidin-1-yl)-3- | | | | | d), 2.33-2.43(2H, m), 2.67-2.78(2H, m), |
| | fluorobenzoic acid | | | | | 3.14(2H, t), 3.23-3.32(2H, m), |
| | | | | | | 4.35-4.46(1H, m), 6.87-7.14(6H, m) |
| 58 | 2-(4-{[4-(4-chloro-2- | A | 511/513 | | | 1.38-1.53(2H, m), 1.65-1.88(5H, m), |
| | methylphenoxy)piperidin-1- | | | | | 1.97-2.08(2H, m), 2.21(3H, s), 2.30(2H, |
| | yl]methyl}piperidin-1-yl)-5- | | | | | d), 2.34-2.44(2H, m), 2.68-2.87(4H, m), |
| | (trifluoromethyl)benzoic | | | | | 3.63(2H, d), 4.36-4.45(1H, m), 6.90(1H, |
| | acid | | | | | d), 7.01-7.14(3H, m), 7.44(1H, dd), |
| | | | | | | 7.62(1H, d) |
| 59 | 2-(4-{[4-(4-chloro-2- | A | 488/490 | | | 1.36-1.51(2H, m), 1.77-1.89(5H, m), |
| | methylphenoxy)piperidin-1- | | | | | 1.97-2.08(2H, m), 2.20(3H, s), 2.30(2H, |
| | yl]methyl}piperidin-1-yl)-5- | | | | | d), 2.33-2.45(2H, m), 2.68-2.79(2H, m), |
| | nitrobenzoic acid | | | | | 2.94-3.05(2H, m), 3.80(2H, d), |
| | | | | | | 4.37-4.45(1H, m), 6.90(1H, d), 6.99(1H, d), |
| | | | | | | 7.07-7.14(2H, m), 8.06(1H, dd), 8.24(1H, d) |
| 60 | 2-(4-{[4-(4-chloro-2- | A | 473/475 | | | 1.29-1.47(2H, m), 1.57-1.76(3H, m), |
| | methylphenoxy)piperidin-1- | | | | | 1.77-1.89(2H, m), 1.97-2.08(2H, m), |
| | yl]methyl}piperidin-1-yl)-3- | | | | | 2.20(3H, s), 2.28(2H, d), 2.33-2.43(2H, |
| | methoxybenzoic acid | | | | | m), 2.67-2.78(2H, m), 3.13-3.23(4H, m), |
| | | | | | | 3.82(3H, s), 4.36-4.45(1H, m), 6.84(2H, |
| | | | | | | d), 6.90(1H, d), 6.99(1H, dd), |
| | | | | | | 7.08-7.13(2H, m) |
| 61 | 2-(4-{[4-(4-chloro-2- | A | 511/513 | | | 1.40-1.56(2H, m), 1.64-1.75(1H, m), |
| | methylphenoxy)piperidin-1- | | | | | 1.79-1.89(4H, m), 1.97-2.08(2H, m), |
| | yl]methyl}piperidin-1-yl)-4- | | | | | 2.21(3H, s), 2.31(2H, d), 2.35-2.44(2H, |
| | (trifluoromethyl)benzoic | | | | | m), 2.69-2.81(4H, m), 3.56(2H, d), |
| | acid | | | | | 4.37-4.45(1H, m), 6.90(1H, d), 7.08-7.16(4H, |
| | | | | | | m), 7.46(1H, d) |
| 62 | 4-chloro-2-(4-{[4-(4-chloro- | A | 477/479 | | | 1.38-1.49(2H, m), 1.59-1.71(1H, m), |
| | 2-methylphenoxy)piperidin- | | | | | 1.75-1.85(4H, m), 1.96-2.04(2H, m), |
| | 1-yl]methyl}piperidin-1- | | | | | 2.18(3H, s), 2.27(2H, d), 2.32-2.39(2H, |
| | yl)benzoic acid | | | | | m), 2.64-2.74(4H, m), 3.45-3.52(2H, m), |
| | | | | | | 4.34-4.43(1H, m), 6.81(1H, dd), |
| | | | | | | 6.86-6.89(2H, m), 7.06(1H, d), 7.08-7.10(1H, |
| | | | | | | m), 7.30(1H, d) |
| 63 | 2-(4-{[4-(3,4- | B | 479/481 | | | 1.30-1.40(1H, m), 1.69(2H, d), |
| | dichlorophenoxy)piperidin- | | | | | 1.76-1.90(4H, m), 1.96-2.06(2H, m), 2.43(2H, s), |
| | 1-yl]methyl}-4- | | | | | 2.52-2.60(2H, m), 2.88-2.95(2H, m), |
| | hydroxypiperidin-1- | | | | | 3.05-3.13(2H, m), 3.21-3.28(2H, m), |
| | yl)benzoic acid | | | | | 4.35-4.43(1H, m), 6.86-6.94(2H, m), |
| | | | | | | 7.01(1H, d), 7.11(1H, d), 7.18-7.24(1H, |
| | | | | | | m), 7.35-7.43(2H, m) |
| 64 | 2-(4-{[4-(4-chloro-2- | A | 461/463 | | | 1.37-1.53(2H, m), 1.57-1.71(1H, m), |
| | methylphenoxy)piperidin-1- | | | | | 1.75-1.87(4H, m), 1.96-2.08(2H, m), |
| | yl]methyl}piperidin-1-yl)-5- | | | | | 2.20(3H, s), 2.29(2H, d), 2.33-2.43(2H, |
| | fluorobenzoic acid | | | | | m), 2.61-2.77(4H, m), 3.41(2H, d), |
| | | | | | | 4.36-4.45(1H, m), 6.88-7.13(6H, m) |
| 65 | 3-chloro-2-(4-{[4-(4-chloro- | A | 477/479 | | | 1.34-1.48(2H, m), 1.64-1.88(5H, m), |
| | 2-methylphenoxy)piperidin- | | | | | 1.97-2.08(2H, m), 2.20(3H, s), 2.30(2H, |
| | 1-yl]methyl}piperidin-1- | | | | | d), 2.33-2.44(2H, m), 2.67-2.77(2H, m), |
| | yl)benzoic acid | | | | | 3.19-3.26(4H, m), 4.36-4.45(1H, m), |
| | | | | | | 6.87-6.98(2H, m), 7.07-7.13(2H, m), |
| | | | | | | 7.18-7.30(2H, m) |
| 66 | 5-chloro-2-(4-{[4-(4-chloro- | A | 477/479 | | | 1.37-1.53(2H, m), 1.59-1.73(1H, m), |
| | 2-methylphenoxy)piperidin- | | | | | 1.77-1.88(4H, m), 1.97-2.07(2H, m), |
| | 1-yl]methyl}piperidin-1- | | | | | 2.20(3H, s), 2.29(2H, d), 2.33-2.43(2H, |
| | yl)benzoic acid | | | | | m), 2.64-2.77(4H, m), 3.44-3.51(2H, m), |
| | | | | | | 4.36-4.45(1H, m), 6.92(2H, dd), |
| | | | | | | 7.07-7.19(3H, m), 7.30(1H, d) |
| 67 | 2-(4-{[4-(4-chloro-3- | A | 509/511 | | | 1.40-1.55(2H, m), 1.63-1.88(5H, m), |
| | methylphenoxy)piperidin-1- | | −ve | | | 1.96-2.07(2H, m), 2.27-2.38(7H, m), |
| | yl]methyl}piperidin-1-yl)-4- | | | | | 2.70-2.81(4H, m), 3.56(2H, d), |
| | (trifluoromethyl)benzoic | | | | | 4.32-4.41(1H, m), 6.76(1H, dd), 6.87(1H, d), |
| | acid | | | | | 7.11-7.17(2H, m), 7.22(1H, d), 7.46(1H, d) |
| 68 | 2-(4-{[4-(4-chloro-3- | A | 459/461 | | | 1.32-1.47(2H, m), 1.60-1.85(5H, m), |
| | methylphenoxy)piperidin-1- | | −ve | | | 1.96-2.07(2H, m), 2.26-2.37(7H, m), |
| | yl]methyl}piperidin-1-yl)-3- | | | | | 2.71-2.81(2H, m), 3.08-3.20(2H, m), |
| | fluorobenzoic acid | | | | | 3.23-3.31(2H, m), 4.32-4.41(1H, m), |
| | | | | | | 6.73-6.79(1H, m), 6.86-7.07(4H, m), |
| | | | | | | 7.20-7.25(1H, m) |
| 69 | 2-(4-{[4-(4-chloro-3- | A | 459/461 | | | 1.38-1.53(2H, m), 1.57-1.70(1H, m), |
| | methylphenoxy)piperidin-1- | | −ve | | | 1.74-1.85(4H, m), 1.96-2.07(2H, m), |
| | yl]methyl}piperidin-1-yl)-5- | | | | | 2.27-2.39(7H, m), 2.61-2.81(4H, m), |
| | fluorobenzoic acid | | | | | 3.37-3.44(2H, m), 4.32-4.42(1H, m), |
| | | | | | | 6.76(1H, dd), 6.86-7.06(4H, m), 7.22(1H, |
| | | | | | | d) |
| 70 | 2-(4-{[4-(4-chloro-3- | A | 509/511 | | | 1.39-1.55(2H, m), 1.65-1.87(5H, m), |
| | methylphenoxy)piperidin-1- | | −ve | | | 1.96-2.07(2H, m), 2.28-2.39(7H, m), |
| | yl]methyl}piperidin-1-yl)-5- | | | | | 2.71-2.87(4H, m), 3.59-3.68(2H, m), |
| | (trifluoromethyl)benzoic | | | | | 4.31-4.42(1H, m), 6.74-6.80(1H, m), |
| | acid | | | | | 6.86-6.89(1H, m), 7.01-7.06(1H, m), |
| | | | | | | 7.20-7.25(1H, m), 7.41-7.47(1H, m), |
| | | | | | | 7.60-7.63(1H, m) |
| 71 | 2-(4-{[4-(4-chloro-2- | A | 455/457 | | | 1.35-1.52(2H, m), 1.55-1.69(1H, m), |
| | methylphenoxy)piperidin-1- | | −ve | | | 1.74-1.88(4H, m), 1.95-2.07(2H, m), |
| | yl]methyl}piperidin-1-yl)-5- | | | | | 2.17-2.43(10H, m), 2.57-2.74(4H, m), |
| | methylbenzoic acid | | | | | 3.40-3.49(2H, m), 4.34-4.44(1H, m), |
| | | | | | | 6.84-6.91(2H, m), 6.98-7.04(1H, m), |
| | | | | | | 7.05-7.11(2H, m), 7.18-7.22(1H, m) |
| 72 | 5-chloro-2-(4-{[4-(4-chloro- | A | 475/477 | | | 1.38-1.53(2H, m), 1.58-1.70(1H, m), |
| | 3-methylphenoxy)piperidin- | | −ve | | | 1.74-1.85(4H, m), 1.95-2.07(2H, m), |
| | 1-yl]methyl}piperidin-1- | | | | | 2.27-2.39(7H, m), 2.64-2.81(4H, m), |
| | yl)benzoic acid | | | | | 3.43-3.52(2H, m), 4.32-4.41(1H, m), |
| | | | | | | 6.74-6.79(1H, m), 6.86-6.89(1H, m), |
| | | | | | | 6.91-6.96(1H, m), 7.13-7.18(1H, m), |
| | | | | | | 7.20-7.25(1H, m), 7.29-7.31(1H, m) |
| 73 | 2-(4-{[4-(4-chloro-3- | A | 455/457 | | | 1.38-1.52(2H, m), 1.56-1.69(1H, m), |
| | methylphenoxy)piperidin-1- | | −ve | | | 1.72-1.84(4H, m), 1.96-2.06(2H, m), |
| | yl]methyl}piperidin-1-yl)-5- | | | | | 2.23-2.38(10H, m), 2.61-2.80(4H, m), |
| | methylbenzoic acid | | | | | 3.44(2H, d), 4.30-4.40(1H, m), 6.76(1H, |
| | | | | | | dd), 6.86-6.89(2H, m), 7.00(1H, dd), |
| | | | | | | 7.16(1H, d), 7.22(1H, d) |
| 74 | 4-chloro-2-(4-{[4-(4-chloro- | A | 475/477 | | | 1.37-1.54(2H, m), 1.61-1.85(5H, m), |
| | 3-methylphenoxy)piperidin- | | −ve | | | 1.96-2.08(2H, m), 2.26-2.39(7H, m), |
| | 1-yl]methyl}piperidin-1- | | | | | 2.64-2.82(4H, m), 3.46-3.54(2H, m), |
| | yl)benzoic acid | | | | | 4.32-4.41(1H, m), 6.73-6.79(1H, m), |
| | | | | | | 6.82-6.91(3H, m), 7.20-7.25(1H, m), |
| | | | | | | 7.31-7.34(1H, m) |
| 75 | 3-chloro-2-(4-{[4-(4-chloro- | A | 475/477 | | | 1.33-1.49(2H, m), 1.63-1.85(5H, m), |
| | 3-methylphenoxy)piperidin- | | −ve | | | 1.96-2.07(2H, m), 2.25-2.39(7H, m), |
| | 1-yl]methyl}piperidin-1- | | | | | 2.71-2.82(2H, m), 3.18-3.26(4H, m), |
| | yl)benzoic acid | | | | | 4.32-4.42(1H, m), 6.74-6.79(1H, m), |
| | | | | | | 6.88(1H, d), 6.95(1H, t), 7.18-7.30(3H, |
| | | | | | | m) |
| 76 | 2-(4-{[4-(4-chloro-3- | A | 473/475 | | | 1.27-1.45(2H, m), 1.59-1.84(5H, m), |
| | methylphenoxy)piperidin-1- | | | | | 1.95-2.07(2H, m), 2.23-2.39(7H, m), |
| | yl]methyl}piperidin-1-yl)-3- | | | | | 2.67-2.81(2H, m), 3.16-3.22(4H, m), |
| | methoxybenzoic acid | | | | | 3.82(3H, s), 4.31-4.42(1H, m), |
| | | | | | | 6.73-6.89(4H, m), 6.96-7.03(1H, m), |
| | | | | | | 7.19-7.25(1H, m) |
| 77 | 2-(4-{[4-(3-chloro-4- | A | 470/472 | | | 1.35-1.50(2H, m), 1.58-1.72(1H, m), |
| | cyanophenoxy)piperidin-1- | | −ve | | | 1.76-1.89(4H, m), 2.00-2.11(2H, m), |
| | yl]methyl}piperidin-1-yl)-6- | | | | | 2.29(2H, d), 2.38(2H, t), 2.65-2.80(4H, |
| | fluorobenzoic acid | | | | | m), 3.52-3.59(2H, m), 4.53-4.63(1H, m), |
| | | | | | | 6.67(1H, t), 6.80(1H, d), 7.04-7.18(2H, |
| | | | | | | m), 7.21(1H, d), 7.71(1H, d) |
| 78 | 2-(4-{[4-(3-chloro-4- | A | 482/484 | | | 1.38-1.53(2H, m), 1.58-1.72(1H, m), |
| | cyanophenoxy)piperidin-1- | | −ve | | | 1.76-1.89(4H, m), 2.01-2.11(2H, m), |
| | yl]methyl}piperidin-1-yl)-4- | | | | | 2.31(2H, d), 2.33-2.43(2H, m), |
| | methoxybenzoic acid | | | | | 2.61-2.81(4H, m), 3.46-3.54(2H, m), 3.78(3H, s), |
| | | | | | | 4.53-4.62(1H, m), 6.41-6.48(2H, m), |
| | | | | | | 7.06(1H, dd), 7.20(1H, d), 7.39(1H, d), |
| | | | | | | 7.72(1H, d) |
| 79 | 2-(4-{[4-(3,4- | A | 475/477 | | | 1.23(4H, m), 1.48-1.65(4H, m), |
| | dichlorobenzyl)piperidin-1- | | | | | 1.70-1.81(2H, m), 1.85-1.98(2H, m), 2.24(5H, |
| | yl]methyl}piperidin-1-yl)-5- | | | | | d), 2.55(2H, d), 2.58-2.70(2H, m), |
| | methylbenzoic acid | | | | | 2.89-2.97(2H, m), 3.38-3.47(2H, m), 6.86(1H, |
| | | | | | | d), 6.96-7.01(1H, m), 7.09-7.17(2H, m), |
| | | | | | | 7.35(1H, d), 7.42(1H, d) |
| 80 | 2-(4-{[4-(4- | A | 441/443 | | | 1.26-1.67(8H, m), 1.72-1.82(2H, m), |
| | chlorobenzyl)piperidin-1- | | | | | 1.86-1.97(2H, m), 2.21-2.28(5H, m), |
| | yl]methyl}piperidin-1-yl)-5- | | | | | 2.55(2H, d), 2.59-2.69(2H, m), |
| | methylbenzoic acid | | | | | 2.88-2.97(2H, m), 3.39-3.47(2H, m), 6.86(1H, |
| | | | | | | d), 6.96-7.01(1H, m), 7.13-7.19(3H, m), |
| | | | | | | 7.24-7.27(1H, m), 7.27-7.30(1H, m) |
| 81 | 2-(4-{[4-(4- | B | 457/459 | | | 1.16-1.34(4H, m), 1.44-1.62(6H, m), |
| | chlorobenzyl)piperidin-1- | | | | | 2.10(2H, d), 2.43(2H, d), 2.75-2.86(2H, |
| | yl]methyl}piperidin-1-yl)-3- | | | | | m), 3.01-3.14(4H, m), 3.70(3H, s), |
| | methoxybenzoic acid | | | | | 6.68-6.77(2H, m), 6.87(1H, t), 7.01-7.09(2H, |
| | | | | | | m), 7.12-7.20(2H, m) |
| 82 | 2-(4-{[4-(3,4- | B | 491/493 | 1.94 | Standard | 1.15-1.33(4H, m), 1.44-1.62(6H, m), |
| | dichlorobenzyl)piperidin-1- | | | | | 1.73-1.86(2H, m), 2.11(2H, d), 2.44(2H, |
| | yl]methyl}piperidin-1-yl)-3- | | | | | d), 2.73-2.88(2H, m), 3.00-3.10(4H, m), |
| | methoxybenzoic acid | | | | | 3.69(3H, s), 6.67-6.75(2H, m), |
| | | | | | | 6.82-6.90(1H, m), 6.97-7.03(1H, m), |
| | | | | | | 7.22-7.25(1H, m), 7.28-7.33(1H, m) |
| 83 | 2-(4-{[4-(4- | B | 425/427 | 2.33 | Standard | 1.24-1.63(8H, m), 1.70-1.77(2H, m), |
| | fluorobenzyl)piperidin-1- | | | | | 1.83-1.92(2H, m), 2.18-2.23(5H, m), |
| | yl]methyl}piperidin-1-yl)-5- | | | | | 2.51(2H, d), 2.57-2.65(2H, m), |
| | methylbenzoic acid | | | | | 2.86-2.93(2H, m), 3.36-3.43(2H, m), 6.83(1H, |
| | | | | | | d), 6.93-6.99(3H, m), 7.10-7.16(3H, m) |
| 84 | 2-(4-{[4-(4- | B | | | | 1.26-1.40(5H, m), 1.55-1.69(5H, m), |
| | fluorobenzyl)piperidin-1- | | | | | 1.83-1.91(2H, m), 2.19(2H, d), 2.51(2H, |
| | yl]methyl}piperidin-1-yl)-3- | | | | | d), 2.86-2.93(2H, m), 3.10-3.18(4H, m), |
| | methoxybenzoic acid | | | | | 3.78(3H, s), 6.78-6.82(2H, m), |
| | | | | | | 6.93-6.99(3H, m), 7.11-7.16(2H, m) |
| 85 | 2-(4-{[3-(3,4- | A | 451/453 | | | 1.39-1.53(3H, m), 1.68-1.78(2H, m), |
| | dichlorophenoxy)azetidin-1- | | −ve | | | 2.49-2.53(2H, m), 2.58-2.69(2H, m), |
| | yl]methyl}piperidin-1-yl)-5- | | | | | 3.17-3.24(2H, m), 3.36-3.43(2H, m), |
| | fluorobenzoic acid | | | | | 3.80-3.87(2H, m), 4.80-4.86(1H, m), |
| | | | | | | 6.80(1H, dd), 6.87-7.05(4H, m), 7.42(1H, |
| | | | | | | d) |
| 86 | 2-(4-{[3-(3,4- | A | 433/435 | | | 1.38-1.56(3H, m), 1.69-1.78(2H, m), |
| | dichlorophenoxy)azetidin-1- | | −ve | | | 2.51(2H, s), 2.67(2H, t), 3.17-3.24(2H, |
| | yl]methyl}piperidin-1- | | | | | m), 3.45-3.53(2H, m), 3.79-3.87(2H, m), |
| | yl)benzoic acid | | | | | 4.78-4.87(1H, m), 6.77-6.97(3H, m), |
| | | | | | | 6.99-7.02(1H, m), 7.12-7.21(1H, m), |
| | | | | | | 7.33(1H, d), 7.41(1H, d) |
| 87 | 2-(4-{[(3S)-3-(4- | A | 413/415 | | | 1.40-1.55(2H, m), 1.55-1.66(1H, m), |
| | chlorophenoxy)pyrrolidin-1- | | −ve | | | 1.78-1.87(2H, m), 1.93-1.99(1H, m), |
| | yl]methyl}piperidin-1- | | | | | 2.27-2.85(8H, m), 2.91-2.98(1H, m), |
| | yl)benzoic acid | | | | | 3.46-3.54(2H, m), 4.84-4.91(1H, m), |
| | | | | | | 6.84-6.91(3H, m), 6.97(1H, d), |
| | | | | | | 7.15-7.22(1H, m), 7.26(2H, dd), 7.34(1H, dd) |
| 88 | 2-(4-{[(3S)-3-(4- | A | 431/433 | | | 1.39-1.53(2H, m), 1.55-1.65(1H, m), |
| | chlorophenoxy)pyrrolidin-1- | | −ve | | | 1.77-1.86(2H, m), 1.88-1.99(1H, m), |
| | yl]methyl}piperidin-1-yl)-5- | | | | | 2.27-2.50(3H, m), 2.53-2.72(3H, m), |
| | fluorobenzoic acid | | | | | 2.74-2.85(2H, m), 2.90-2.97(1H, m), |
| | | | | | | 3.37-3.44(2H, m), 4.84-4.92(1H, m), |
| | | | | | | 6.86-7.05(5H, m), 7.26(2H, dd) |
| 89 | 2-(4-{[3-(3-chloro-4- | A | 424/426 | | | 1.38-1.58(3H, m), 1.69-1.81(2H, m), |
| | cyanophenoxy)azetidin-1- | | −ve | | | 2.49-2.56(2H, m), 2.61-2.74(2H, m), |
| | yl]methyl}piperidin-1- | | | | | 3.21-3.30(2H, m), 3.44-3.54(2H, m), |
| | yl)benzoic acid | | | | | 3.81-3.91(2H, m), 4.91-5.00(1H, m), |
| | | | | | | 6.84-7.00(3H, m), 7.10-7.23(2H, m), |
| | | | | | | 7.31-7.38(1H, m), 7.71-7.78(1H, m) |
| 90 | 2-(4-{[4-(3-chloro-4-cyano- | A | 466/468 | | | 1.39-1.54(2H, m), 1.59-1.74(1H, m), |
| | 2-methylphenoxy)piperidin- | | −ve | | | 1.77-1.93(4H, m), 2.03-2.13(2H, m), |
| | 1-yl]methyl}piperidin-1- | | | | | 2.29-2.35(5H, m), 2.39-2.49(2H, m), |
| | yl)benzoic acid | | | | | 2.64-2.76(4H, m), 3.47-3.55(2H, m), |
| | | | | | | 4.59-4.67(1H, m), 6.88(1H, t), 6.97(1H, |
| | | | | | | d), 7.10(1H, d), 7.20(1H, dd), 7.35(1H, |
| | | | | | | dd), 7.63(1H, d) |
| 91 | 2-(4-{[3-(3-chloro-4- | A | 442/444 | | | 1.12-1.27(2H, m), 1.71-1.89(4H, m), |
| | cyanophenoxy)azetidin-1- | | −ve | | | 1.99-2.10(2H, m), 2.25(2H, d), |
| | yl]methyl}piperidin-1-yl)-5- | | | | | 2.31-2.41(3H, m), 2.63-2.78(4H, m), 3.13(2H, d), |
| | fluorobenzoic acid | | | | | 4.51-4.62(1H, m), 7.05(1H, dd), 7.21(1H, |
| | | | | | | d), 7.70(1H, d) |
| 92 | 2-(4-{[4-(3,4- | D | 462/464 | 1.36 | Standard | 1.20-1.32(3H, m), 1.65-1.76(4H, m), |
| | dichlorophenoxy)piperidin- | | −ve | | | 1.93-2.01(2H, m), 2.20(2H, d), |
| | 1-yl]methyl}piperidin-1- | | | | | 2.22-2.30(2H, m), 2.65-2.73(2H, m), 2.81(2H, t), |
| | yl)nicotinic acid | | | | | 4.00(2H, d), 4.37-4.44(1H, m), 6.64(1H, |
| | | | | | | dd), 6.95(1H, dd), 7.16(1H, d), 7.44(1H, |
| | | | | | | d), 7.57(1H, dd), 7.99(1H, dd) |
| 93 | 2-[4-({4[4- | A | 488/490 | | | 1.35-1.50(2H, m), 1.58-1.72(1H, m), |
| | (aminocarbonyl)-3- | | −ve | | | 1.76-1.87(4H, m), 1.99-2.10(2H, m), |
| | chlorophenoxy]piperidin-1- | | | | | 2.27-2.41(4H, m), 2.66-2.80(4H, m), |
| | yl}methyl)piperidin-1-yl]-6- | | | | | 3.51-3.60(2H, m), 4.44-4.54(1H, m), |
| | fluorobenzoic acid | | | | | 6.67(1H, t), 6.81(1H, d), 6.96(1H, dd), |
| | | | | | | 7.03(1H, d), 7.14(1H, td), 7.52(1H, d) |
| 94 | 3-(4-{[4-(3,4- | A | 463/ | | | 1.36-1.45(2H, m), 1.73-1.86(3H, m), |
| | dichlorophenoxy)piperidin- | | 467(M+H) | | | 1.91-1.95(2H, m), 2.03-2.08(2H, m), |
| | 1-yl]methyl}piperidin-1- | | | | | 2.31-2.33(2H, d), 2.36-2.40(2H, t), |
| | yl)benzoic acid | | | | | 2.72-2.77(4H, m), 3.73-3.76(2H, d), |
| | | | | | | 4.38-4.48(1H, m), 6.91-6.94(1H, dd), |
| | | | | | | 7.08-7.12(2H, m), 7.24-7.28(1H, t), 7.40-7.43(1H, |
| | | | | | | d), 7.47-7.49(1H, d), 7.67(1H, s). |
| 95 | 3-(4-{[4-(4- | A | 429/ | | | δ_{(CDCL3)} 1.44(2H, q), 1.81-2.10(5H, m), |
| | chlorophenoxy)piperidin-1- | Isolated as | 431(M+H) | | | 2.26-2.38(2H, m), 2.59(2H, d), 2.74(2H, |
| | yl]methyl}piperidin-1- | impurity in | | | | t), 2.84-3.02(4H, m), 3.73(2H, d), |
| | yl)benzoic acid | example 95 | | | | 4.52(1H, s), 6.83(2H, d), 7.00-7.07(1H, m), |
| | | | | | | 7.16-7.29(3H, m), 7.50(1H, d), 7.62(1H, |
| | | | | | | s) |
| 96 | 3-(4-{[4-(3,4-dichloro-2- | A | 477/ | | | 1.29-1.49(2H, m), 1.70-1.98(5H, m), |
| | methylphenoxy)piperidin-1- | | 481(M+H) | | | 2.01-2.12(2H, m), 2.32(2H, d), 2.35(3H, |
| | yl]methyl}piperidin-1- | | | | | s), 2.38-2.48(2H, m), 2.74(4H, t), |
| | yl)benzoic acid | | | | | 3.74(2H, d), 4.43-4.53(1H, m), 6.96(1H, d), |
| | | | | | | 7.09(1H, d), 7.29(2H, mult), 7.49(1H, d), |
| | | | | | | 7.67(1H, s) |
| 97 | 3-(4-{[4-(3,4- | C | 477/479 | | | 1.18-1.37(3H, m), 1.56-1.74(3H, m), |
| | dichlorophenoxy)piperidin- | | | | | 1.74-1.82(2H, m), 1.88-1.97(2H, m), |
| | 1-yl]methyl}piperidin-1-yl)- | | | | | 2.20(3H, s), 2.21-2.30(3H, m), |
| | 4-methylbenzoic acid | | | | | 2.54-2.70(4H, m), 2.99-3.06(2H, m), |
| | | | | | | 4.26-4.35(1H, m), 6.80(1H, dd), 7.00(1H, d), |
| | | | | | | 7.03(1H, d), 7.28(1H, d), 7.44(1H, dd), |
| | | | | | | 7.59(1H,d) |
| 98 | 3-(4-{[4-(3,4- | C | 477/479 | | | 1.26-1.46(3H, m), 1.62-1.73(1H, m), |
| | dichlorophenoxy)piperidin- | | | | | 1.73-1.89(4H, m), 1.97-2.07(2H, m), |
| | 1-yl]methyl}piperidin-1-yl)- | | | | | 2.27-2.33(3H, m), 2.36(3H, s), 2.62(2H, |
| | 2-methylbenzoic acid | | | | | t), 2.71-2.80(2H, m), 3.05-3.12(2H, m), |
| | | | | | | 4.36-4.44(1H, m), 6.89(1H, dd), 6.97(1H, |
| | | | | | | dd), 7.02-7.08(2H, m), 7.10(1H, d), |
| | | | | | | 7.38(1H, d) |
| 99 | 3-(4-{[4-(3,4- | E | 477/479 | 0.49 | fast | 1.17-1.33(3H, m), 1.53-1.73(3H, m), |
| | dichlorophenoxy)piperidin- | | | | | 1.75-1.82(2H, m), 1.87-1.96(2H, m), |
| | 1-yl]methyl}piperidin-1-yl)- | | | | | 2.15-2.28(6H, m), 2.55-2.69(4H, m), |
| | 5-methylbenzoic acid | | | | | 3.55-3.62(2H, m), 4.25-4.34(1H, m), |
| | | | | | | 6.77-6.81(2H, m), 7.00(1H, d), |
| | | | | | | 7.18-7.20(1H, m), 7.28(1H, d), 7.33(1H, s) |
| 100 | 4-chloro-3-(4-{[4-(3,4- | E | 498/500 | | | 1.37-1.49(2H, m), 1.65-1.83(3H, m), |
| | dichlorophenoxy)piperidin- | | | | | 1.84-1.91(2H, m), 1.97-2.06(2H, m), |
| | 1-yl]methyl}piperidin-1- | | | | | 2.28-2.39(4H, m), 2.64-2.79(4H, m), |
| | yl)benzoic acid | | | | | 3.33-3.41(2H, m), 4.36-4.44(1H, m), |
| | | | | | | 6.89(1H, dd), 7.08(1H, d), 7.31(1H, d), |
| | | | | | | 7.38(1H, d), 7.55(1H, dd), 7.74(1H, d) |
| 101 | 3-(4-{[4-(3,4- | E | 493/495 | 1.46 | Standard | 1.38-1.50(2H, m), 1.64-1.83(3H, m), |
| | dichlorophenoxy)piperidin- | | | | | 1.83-1.90(2H, m), 1.97-2.06(2H, m), |
| | 1-yl]methyl}piperidin-1-yl)- | | | | | 2.27-2.38(4H, m), 2.57-2.66(2H, m), 2.71-2.80(2H, |
| | 4-methoxybenzoic acid | | | | | m), 3.40-3.46(2H, m), 3.89(3H, s), |
| | | | | | | 4.36-4.43(1H, m), 6.87-6.91(1H, m), 6.92(1H, |
| | | | | | | d), 7.10(1H, d), 7.37(1H, d), 7.64-7.70(2H, |
| | | | | | | m) |
| 102 | 3-(4-{[4-(2,4-dichloro-3- | A | | | | 1.27-1.44(2H, m), 1.67-1.77(1H, m), |
| | methylphenoxy)piperidin-1- | | | | | 1.80-1.94(4H, m), 1.96-2.06(2H, m), 2.29(2H, |
| | yl]methyl)piperidin-1- | | | | | d), 2.35-2.43(2H, m), 2.45(3H, s), |
| | yl)benzoic acid | | | | | 2.65-2.80(4H, m), 3.71(2H, d), 4.42-4.51(1H, |
| | | | | | | m), 6.95(1H, d), 7.05(1H, dd), |
| | | | | | | 7.19-7.27(2H, m), 7.44(1H, d), 7.63(1H, t) |
| 103 | 2-chloro-5-(4-{[4-(3,4- | E | 497/499 | 1.47 | Standard | 1.24-1.40(2H, m), 1.60-1.82(3H, m), |
| | dichlorophenoxy)piperidin- | | | | | 1.82-1.91(2H, m), 1.96-2.05(2H, m), |
| | 1-yl]methyl}piperidin-1- | | | | | 2.24-2.29(2H, m), 2.29-2.38(2H, m), 2.64-2.78(4H, |
| | yl)benzoic acid | | | | | m), 3.62-3.69(2H, m), 4.35-4.43(1H, m), |
| | | | | | | 6.87(1H, dd), 6.89(1H, dd), 7.04(1H, d), |
| | | | | | | 7.09(1H, d), 7.15(1H, d), 7.37(1H, d) |
| 104 | 6-(4-{[4-(3,4- | E | 464/466 | 1.47 | Standard | 1.16-1.30(2H, m), 1.72-1.90(5H, m), |
| | dichlorophenoxy)piperidin- | | | | | 1.96-2.05(2H, m), 2.23-2.29(2H, m), |
| | 1-yl]methyl}piperidin-1- | | | | | 2.29-2.38(2H, m), 2.70-2.86(4H, m), |
| | yl)pyridine-2-carboxylic acid | | | | | 4.35-4.45(3H, m), 6.80(1H, d), 6.89(1H, |
| | | | | | | dd), 7.09(1H, d), 7.25(1H, d), 7.37(1H, d), |
| | | | | | | 7.54(1H, dd) |
| 105 | 5-(4-{[4-(3,4- | E | 478/480 | 1.31 | Standard | 1.28-1.40(2H, m), 1.68-1.83(3H, m), |
| | dichlorophenoxy)piperidin- | | | | | 1.85-1.93(2H, m), 1.97-2.05(2H, m), |
| | 1-yl]methyl}piperidin-1-yl)- | | | | | 2.26-2.30(2H, m), 2.30-2.38(2H, m), |
| | 2-methylnicotinic acid | | | | | 2.54-2.57(3H, m), 2.69-2.79(4H, m), |
| | | | | | | 3.65-3.73(2H, m), 4.35-4.43(1H, m), |
| | | | | | | 6.89(1H, dd), 7.10(1H, d), 7.37(1H, d), |
| | | | | | | 7.49(1H, d), 8.02(1H, d) |
| 106 | 3-(4-{[4-(3,4- | D | | | | 1.15-1.32(4H, m), 1.40-1.66(4H, m), |
| | dichlorobenzyl)piperidin-1- | | | | | 1.73-1.86(4H, m), 2.13(2H, d), 2.44(2H, |
| | yl]methyl}piperidin-1- | | | | | d), 2.55-2.64(2H, m), 2.79-2.86(2H, m), |
| | yl)benzoic acid | | | | | 3.56-3.63(2H, m), 6.91-6.97(1H, m), |
| | | | | | | 7.00(1H, dd), 7.11(1H, t), 7.24(1H, d), |
| | | | | | | 7.28-7.36(2H, m), 7.52(1H, s) |

Hydrolysis methods:
A, LiOH/aq THF or aq MeOH or aq THF - MeOH
B, KOTMS/THF/RT 3 days
C, TFA/dichloromethane
D, 6M HCl aq reflux
E, 4M HCl/dioxan

### EXAMPLE 107

Pharmacological Analysis: Calcium flux [Ca²⁺]ᵢ assay

Human Eosinophils

Human eosinophils were isolated from EDTA anticoagulated peripheral blood as previously described (Hansel et al., J. Immunol. Methods, 1991, 145, 105-110). The cells were resuspended (5×10⁶ ml⁻¹) and loaded with 5 μM FLUO-3/AM+Pluronic F127 2.2 μl/ml (Molecular Probes) in low potassium solution (LKS; NaCl 118 mM, MgSO₄ 0.8 mM, glucose 5.5 mM, Na₂CO₃ 8.5 mM, KCl 5 mM, HEPES 20 mM, CaCl₂ 1.8 mM, BSA 0.1%, pH 7.4) for one hour at room temperature. After loading, cells were centrifuged at 200 g for 5 min and resuspended in LKS at 2.5×10⁶ ml⁻¹. The cells were then transferred to 96 well FLIPr plates (Poly-D-Lysine plates from Becton Dickinson pre-incubated with 5 μM fibronectin for two hours) at 25 μ/well. The plate was centrifuged at 200 g for 5 min and the cells were washed twice with LKS (200 μl; room temperature).

A compound of the Examples was pre-dissolved in DMSO and added to a final concentration of 0.1%(v/v) DMSO. Assays were initiated by the addition of an A₅₀ concentration of eotaxin and the transient increase in fluo-3 fluorescence (1_{Ex}=490 nm and 1_{Ex}=520 nm) monitored using a FLIPR (Fluorometric Imaging Plate Reader, Molecular Devices, Sunnyvale, U.S.A.).

Compounds of the Examples were found to be antagonists if the increase in fluorescence induced by eotaxin (a selective CCR3 agonist) was inhibited in a concentration dependent manner. The concentration of antagonist required to inhibit the fluorescence by 50% can be used to determine the IC₅₀ for the antagonist at the CCR3 receptor.

### EXAMPLE 108

Human Eosinophil Chemotaxis

Human eosinophils were isolated from EDTA anticoagulated peripheral blood as previously described (Hansel et al., J. Immunol. Methods, 1991, 145, 105-110). The cells were resuspended at 10×10⁶ ml⁻¹ in RPMI containing 200 IU/ml penicillin, 200 μg/ml streptomycin sulfate and supplemented with 10% H[FCS, at room temperature.

Eosinophils (700 μl) were pre-incubated for 15 mins at 37° C. with 7 μl of either vehicle or compound (100× required final concentration in 10% DMSO). The chemotaxis plate (ChemoTx, 3 μm pore, Neuroprobe) was loaded by adding 28 μl of a concentration of eotaxin 0.1 to 100 nM (a selective CCR3 agonist over this concentration range) containing a concentration of a compound according to the Examples or solvent to the lower wells of the chemotaxis plate. The filter was then placed over the wells and 25 μl of eosinophil suspension were added to the top of the filter. The plate was incubated for 1 hr at 37° C. in a humidified incubator with a 95% air/5% CO₂ atmosphere to allow chemotaxis.

The medium, containing cells that had not migrated, was carefully aspirated from above the filter and discarded. The filter was washed once with phosphate buffered saline (PBS) containing 5 mM EDTA to remove any adherent cells. Cells that had migrated through the filter were pelleted by centrifugation (300×g for 5 mins at room temperature) and the filter removed and the supernatant transferred to each well of a 96-well plate (Costar). The pelleted cells were lysed by the addition of 28 μl of PBS containing 0.5% Triton x100 followed by two cycles of freeze/thawing. The cell lysate was then added to the supernatant. The number of eosinophils migrating was quantified according to the method of Strath et al., J. Immunol. Methods, 1985, 83, 209 by measuring eosinophil peroxidase activity in the supernatant.

Compounds of the Examples were found to be antagonists of eotaxin mediated human eosinophil chemotaxis if the concentration response to eotaxin was shifted to the right of the control curve. Measuring the concentration of eotaxin required to give 50% chemotaxis in the presence or absence of compounds enables the apparent affinity of the compounds at CCR3 to be calculated.

### EXAMPLE 109

Guinea-Pig Isolated Trachea

(See for example, Harrison, R. W. S., Carswell, H. & Young, J. M. (1984) European J. Pharmacol., 106, 405-409.)

Male albino Dunkin-Hartley guinea-pigs (250 g) were killed by cervical dislocation and the whole trachea removed. After clearing the adherent connective tissue, the trachea was cut into six ring segments each three cartilage bands wide and then suspended in 20 ml organ baths containing Krebs-Henseleit solution of the following composition (mM): NaCl 117.6, NaH₂PO₄ 0.9, NaHCO₃ 25.0, MgSO₄ 1.2, KCl 5.4, CaCl₂ 2.6 and glucose 11.1. The buffer was maintained at 37° C. and gassed with 5% CO₂ in oxygen. Indomethacin (2.8 μM) was added to the Krebs solution to prevent development of smooth muscle tone due to the synthesis of cyclo-oxygenase products. The tracheal rings were suspended between two parallel tungsten wire hooks, one attached to an Ormed beam isometric force transducer and the other to a stationary support in the organ bath. Changes in isometric force were recorded on 2-channel Sekonic flat bed chart recorders.

Experimental Protocols

At the beginning of each experiment a force of 1 g was applied to the tissues and this was reinstated over a 60 minute equilibration period until a steady resting tone was achieved. Subsequently, a cumulative histamine concentration effect (E/[A]) curve was constructed at 0.5 log₁₀ unit increments, in each tissue. The tissues were then washed and approximately 30 minutes later, test compound or vehicle (20% DMSO) was added. Following an incubation period of 60 minutes a second E/[A] curve was performed to histamine.

Contraction responses were recorded as a percentage of the first curve maximum.

Data Analysis

Experimental E/[A] curve data were analysed for the purposes of estimating the potencies (p[A₅₀] values) of histamine in the absence and presence of the test compound. Affinity (pA₂) values of test compounds were subsequently calculated using the following equation:
log(r−1)=log [B]+pA₂
where r=[A]₅₀ in presence of test compound/[A]₅₀ in absence of antagonist and [B] is the concentration of test compound. Compounds of the Examples were found to be H1 antagonists.

### EXAMPLE 110

Histamine H1 receptor binding activity of compounds of the invention was assessed by competition displacement of 1 nM [3H]-pyrilamine (Amersham, Bucks, Product code TRK 608, specific activity 30 Ci/mmol) to 2 μg membranes prepared from recombinant CHO-K1 cells expressing the human H1 receptor (Euroscreen SA, Brussels, Belgium, product code ES-390-M) in assay buffer (50 mM Tris pH 7.4 containing 2 mM MgCl₂, 250 mM sucrose and 100 mM NaCl) for 1 hour at room temperature.

The following compounds of the invention gave inhibition of [3H] pyrilimine binding:

| Example | H1 pKi |
|---|---|
| 1 | 8.6 |
| 32 | 8.0 |
| 33 | 6.6 |
| 34 | 6.2 |
| 36 | 8.0 |
| 38 | 7.2 |
| 47 | 8.3 |
| 48 | 7.1 |
| 49 | 7.3 |
| 55 | 6.5 |
| 59 | 8.8 |
| 63 | 7.3 |
| 68 | 7.8 |
| 74 | 8.6 |
| 96 | 7.7 |
| 99 | 8.1 |
| 106 | 7.7 |

1. A compound of formula (I): 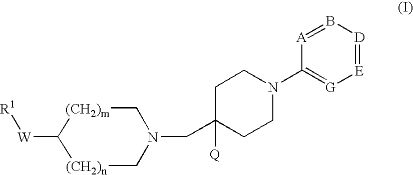
wherein:
n and m are, independently, 0 or 1;
A, B, D, E, G represent one each of CCO₂R⁵, CR², CR³, CR⁴, and CH or N,
Q is hydrogen or hydroxy;
W is CH₂, O, NH or N(C₁₋₄ alkyl);
R¹ is phenyl optionally substituted by halogen, cyano, C₁₋₄ alkyl, C₁₋₄ haloalkyl, C₁₋₄ alkoxy or C₁₋₄ haloalkoxy;
R², R³ and R⁴ are, independently, hydrogen, halogen, cyano, nitro, hydroxy, NR⁶R⁷, C₁₋₆ alkyl (optionally substituted with halogen), C₁₋₆ alkoxy (optionally substituted with halogen), S(O)ₚ(C₁₋₆ alkyl), S(O)_{q}CF₃ or S(O)₂NR⁷R⁸;
R⁵ is hydrogen, C₁₋₆ alkyl or benzyl;
p and q are, independently, 0, 1 or 2;
R⁶, R⁷, R⁸ and R⁹ are, independently, hydrogen, C₁₋₆ alkyl (optionally substituted by halogen, hydroxy or C₃₋₆ cycloalkyl), CH₂(C₂₋₅ alkenyl), phenyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heterocyclyl (itself optionally substituted by halogen, hydroxy, nitro, NH₂, NH(C₁₋₄ alkyl), N(C₁₋₄ alkyl)₂ (and these alkyl groups may join to form a ring as described for R⁶ and R⁷ below), S(O)₂(C₁₋₄ alkyl), S(O)₂NH₂, S(O)₂NH(C₁₋₄ alkyl), S(O)₂N(C₁₋₄ alkyl)₂ (and these alkyl groups may join to form a ring as described for R⁶ and R⁷ below), cyano, C₁₋₄ alkyl, C₁₋₄ alkoxy, C(O)NH₂, C(O)NH(C₁₋₄ alkyl), C(O)N(C₁₋₄ alkyl)₂ (and these alkyl groups may join to form a ring as described for R⁶ and R⁷ below), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
alternatively NR⁶R⁷ or NR⁸R⁹ may, independently, form a 4-7 membered heterocyclic ring,
or an N-oxide thereof; or a pharmaceutically acceptable salt thereof; or a solvate thereof.

2. A compound of formula (I) as claimed in claim 1 wherein W is O.

3. A compound as claimed in claim 1 wherein n and m are both 1.

4. A compound as claimed in claim 1, wherein R¹ is phenyl optionally substituted with halogen, cyano, C₁₋₄ alkyl or C₁₋₄ alkoxy.

5. A compound of formula (I) as claimed in claim 1 wherein Q is hydrogen.

6. A compound of formula (I) as claimed in claim 1 wherein one of A, B, D, E, G represent one each of CCO₂R⁵, CR², CR³, CR⁴, and CH or N; wherein R², R³ and R⁴, are, independently, hydrogen, halogen, cyano, nitro, C₁₋₄ alkyl, C₁₋₄ alkoxy, CF₃, OCF₃, S(O)₂(C₁₋₄ alkyl) or S(O)₂NH₂; and R⁵ is hydrogen or C₁₋₆ alkyl.

7. A process for preparing a first compound of formula (I) as claimed in claim 1, the process comprising:
a. nucleophilic substitution of fluoride (II): 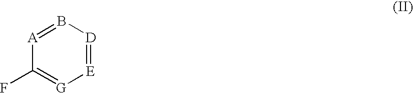
with a compound of formula (III): 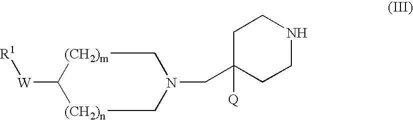
in the presence of a suitable solvent, at a suitable temperature, optionally in the presence of a suitable base;
b. when R⁵ is hydrogen, said first compound may be converted to a second compound of formula (I) as claimed in claim 1 where R⁵ is not hydrogen by a standard esterification method well known in the art;
c. when R⁵ is not hydrogen said compound may be converted to a second compound of formula (I) as claimed in claim 1 where R⁵ is hydrogen by a standard ester hydrolysis method well known in the art; or
d. a Buchwald-Hartwig amination, where a compound of formula (VIII): 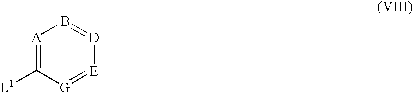
wherein L¹ is bromo or iodo; is reacted with a compound of formula (III): 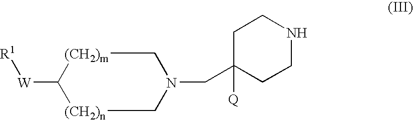
in the presence of a suitable palladium compound, a ligand, a base and a solvent, at a suitable temperature.

8. A pharmaceutical composition which comprises a compound of the formula (I), or a pharmaceutically acceptable salt thereof or solvate thereof as claimed in claim 1, and a pharmaceutically acceptable adjuvant, diluent or carrier.

9-10. (canceled)

11. A method of treating a chemokine mediated disease state in a mammal suffering from, or at risk of, said disease, which comprises administering to a mammal in need of such treatment a therapeutically effective amount of a compound of formula (I), or a pharmaceutically acceptable salt thereof or solvate thereof as claimed in claim 1.

12. The compound as claimed in claim 1, wherein NR⁶R⁷ or NR⁸R⁹, independently, form azetidine, pyrrolidine, piperidine, azepine, morpholine or piperazine, each of which is optionally substituted by C₁₋₄ alkyl on the distal nitrogen.

13. A compound as claimed in claim 2, wherein n and m are both 1.

14. A compound as claimed in claim 2, wherein R¹ is phenyl optionally substituted with halogen, cyano, C₁₋₄ alkyl or C₁₋₄ alkoxy.

15. A compound as claimed in claim 2, wherein Q is hydrogen.

16. A compound as claimed in claim 12, wherein W is 0.

17. A compound as claimed in claim 12, wherein one of A, B, D, E, G represent one each of CCO₂R⁵, CR², CR³, CR⁴, and CH or N; wherein R², R³ and R⁴, are, independently, hydrogen, halogen, cyano, nitro, C₁₋₄ alkyl, C₁₋₄ alkoxy, CF₃, OCF₃, S(O)₂(C₁₋₄ alkyl) or S(O)₂NH₂; and R⁵ is hydrogen or C₁₋₆ alkyl.

18. A compound as claimed in claim 12, wherein n and m are both 1.

19. A compound as claimed in claim 12, wherein R¹ is phenyl optionally substituted with halogen, cyano, C₁₋₄ alkyl or C₁₋₄ alkoxy.

20. A compound as claimed in claim 12, wherein Q is hydrogen.

21. A compound as claimed in claim 12, wherein one of A, B, D, E, G represent one each of CCO₂R⁵, CR², CR³, CR⁴, and CH or N; wherein R², R³ and R⁴, are, independently, hydrogen, halogen, cyano, nitro, C₁₋₄ alkyl, C₁₋₄ alkoxy, CF₃, OCF₃, S(O)₂(C₁₋₄ alkyl) or S(O)₂NH₂; and R⁵ is hydrogen or C₁₋₆ alkyl.
